(19) United States
(12) Patent Application Publication
(10) Pub. No.: US 2021/0015889 A1
(43) Pub. Date: Jan. 21, 2021

(54) METHODS OF DISRUPTING A BIOFILM AND/OR PREVENTING FORMATION OF SAME

(71) Applicants: Yeda Research and Development Co. Ltd., Rehovot (IL); Hadasit Medical Research Services and Development Ltd., Jerusalem (IL)

(72) Inventors: Ilana KOLODKIN-GAL, Rehovot (IL); Alona KEREN-PAZ, Rehovot (IL); Eitan KEREM, Mevaseret-Zion (IL); Gideon ZAMIR, Jerusalem (IL); Dror KOLODKIN-GAL, Rehovot (IL); Malena COHEN-CYMBERKNOH, Mevaseret-Zion (IL)

(73) Assignees: Yeda Research and Development Co. Ltd., Rehovot (IL); Hadasit Medical Research Services and Development Ltd., Jerusalem (IL)

(21) Appl. No.: 17/035,829

(22) Filed: Sep. 29, 2020

Related U.S. Application Data
(63) Continuation of application No. PCT/IL2019/050369, filed on Mar. 28, 2019.

(30) Foreign Application Priority Data
Mar. 29, 2018 (IL) 258467

(51) Int. Cl. A61K 38/14; A61K 31/43; A61K 38/00

(52) U.S. Cl. CPC A61K 38/14; A61K 38/005; A61K 31/43

(57) ABSTRACT

Methods of disrupting a biofilm and/or preventing formation of a biofilm are provided. Accordingly there is provided a method of reducing or preventing formation of a biofilm and/or disrupting a biofilm comprising contacting a biofilm-producing microorganism with a carbonic anhydrase inhibitor; a urease inhibitor; a Ca²⁺ ATPase inhibitor; a tlp activator; and/or a myo-inositol catabolism pathway activator. Also provided are articles of manufacture and methods of treating a medical condition in which disrupting a biofilm and/or preventing formation of same is beneficial and methods of predicting or increasing sensitivity to an anti-microbial agent.

### RELATED APPLICATIONS

This application is a Continuation of PCT Patent Application No. PCT/IL2019/050369 having international filing date of Mar. 28, 2019, which claims the benefit of priority of Israel Patent Application No. 258467 filed on Mar. 29, 2018. The contents of the above applications are all incorporated by reference as if fully set forth herein in their entirety.

### SEQUENCE LISTING STATEMENT

The ASCII file, entitled 84481SequenceListing.txt, created on Sep. 29, 2020, comprising 11,390 bytes, submitted concurrently with the filing of this application is incorporated herein by reference.

### FIELD AND BACKGROUND OF THE INVENTION

The present invention, in some embodiments thereof, relates to methods of disrupting a biofilm and/or preventing formation of same.

Many bacteria are planktonic, namely they move freely around in water and other liquid media, however, many pathogenic and harmful bacteria are or become sessile, namely attached to a surface where they form biofilms—complex differentiated communities held together by an extracellular polymeric substance. In the biofilm, individual cells take part in complex multi-cellular processes using a variety of chemical and metabolic cues to coordinate activity within the community, as well as across species¹⁻³. Biofilms may form on living or non-living surfaces and can be prevalent in natural, industrial and hospital settings. Biofilms provide significant benefits to constituent bacteria e.g. they protect their residents from environmental assaults, and improve their attachment to many different hosts or abiotic surfaces. However, maybe more important is the significant role of biofilms in resistance to treatment. Thus, infections associated with biofilm growth usually are challenging to eradicate, mostly due to their increased antibiotic resistance and tolerance to the immune response as compared with planktonic cells. For example, in a biofilm, microorganisms can be up to 1,000 times more resistant to antibiotics than planktonic bacteria (Bryers, 2008). An example to a lethal chronic infection associated with biofilm formation is Cystic fibrosis (CF), the most common life threatening autosomal recessive disorder among Caucasians, with a rate of one case per 2,500 births. Respiratory infection with Pseudomonas aeruginosa is the most common respiratory system infection that is associated with increased morbidity and mortality in patients with CF [Yoon and Hassett, xpert review of anti-infective therapy (2004) 2: 611-623; Kerem E et al. Eur Respir J. (2013) 43(1):125-33]. Once chronic infection with P. aeruginosa is established, it is almost impossible to eradicate it; therefore, early eradication and treatment is crucial in order to avoid chronic infection and improve CF survival and quality of life [Cohen-Cymberknoh et al., Journal of cystic fibrosis: official journal of the European Cystic Fibrosis Society, (2016) doi:10.1016/j.jcf.2016.04.006; and Cohen-Cymberknoh et al., American journal of respiratory and critical care medicine (2011) 183: 1463-1471]. The persistence of chronic P. aeruginosa lung infection in CF is mainly due to biofilm-growing mucoid (alginate-producing) strains.

The mechanisms supporting this phenotypic resistance, as well as those driving the stages of the transition of bacteria from free-living bacteria to differentiated biofilms are poorly understood. The ability of biofilm-forming bacteria to form complex architectures was mainly attributed to their organic extracellular matrix. Several works have also shown that precipitation of calcium carbonate also contributes to the assembly of the complex biofilm architecture⁸⁻¹³.

Additional background art includes:

Oppenheimer-Shaanan et al. npj Biofilms and Microbiomes (2016) 2: 15031;

Rajendran et al., BMC Microbiol. (2014) 14: 303;

Lovely Rahaman et al. International Journal of Pharmacy and Pharmaceutical Sciences (2018) 10 (3);

Morris N S et al. Urol Res. (1998) 26(4):275-9;

Musk D J Jr et al. J Appl Microbiol. (2008) 105(2): 380-8;

International Patent Application Publication No: WO2004091611;

International Patent Application Publication No: WO2009106211;

International Patent Application Publication No: WO1998050020

US Patent Application Publication No: US20100168808;

US Patent Application Publication No: US20140128399;

US Patent Application Publication No: US20080241275; and

U.S. Pat. No. 9,243,036.

### SUMMARY OF THE INVENTION

According to an aspect of some embodiments of the present invention there is provided a method of reducing or preventing formation of a biofilm and/or disrupting a biofilm, the method comprising contacting a biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when the agent is the carbonic anhydrase inhibitor or the urease inhibitor the at least 1 agent is at least 2 agents, thereby reducing or preventing formation of the biofilm and/or disrupting the biofilm.

According to an aspect of some embodiments of the present invention there is provided a method of reducing or preventing formation of a biofilm and/or disrupting a biofilm, the method comprising contacting a biofilm-producing microorganism with a carbonic anhydrase inhibitor and a urease inhibitor, thereby reducing or preventing formation of the biofilm and/or disrupting the biofilm.

According to an aspect of some embodiments of the present invention there is provided a method of increasing sensitivity of a biofilm-producing bacteria to an anti-microbial agent, the method comprising contacting the biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when the agent is the carbonic anhydrase inhibitor or the urease inhibitor the at least 1 agent is at least 2 agents,

thereby increasing sensitivity of the biofilm-producing bacteria to the anti-microbial agent.

According to an aspect of some embodiments of the present invention there is provided a method of increasing sensitivity of a biofilm-producing bacteria to an anti-microbial agent, the method comprising contacting the biofilm-producing microorganism with a carbonic anhydrase inhibitor and a urease inhibitor, thereby increasing sensitivity of the biofilm-producing bacteria to the anti-microbial agent.

According to some embodiments of the invention, the carbonic anhydrase inhibitor and/or the urease inhibitor is administered at a non-cytotoxic dose to the microorganism.

According to some embodiments of the invention, the agent is administered at a non-cytotoxic dose to the microorganism.

According to an aspect of some embodiments of the present invention there is provided a method of reducing or preventing formation of a biofilm and/or disrupting a biofilm, the method comprising contacting a biofilm-producing bacteria with a carbonic anhydrase inhibitor and/or a urease inhibitor, wherein the carbonic anhydrase inhibitor and/or the urease inhibitor is administered at a non-cytotoxic dose to the microorganism, thereby reducing or preventing formation of the biofilm and/or disrupting the biofilm.

According to an aspect of some embodiments of the present invention there is provided a method of increasing sensitivity of a biofilm-producing bacteria to an anti-microbial agent, the method comprising contacting the biofilm-producing microorganism with a carbonic anhydrase inhibitor and/or a urease inhibitor, wherein the carbonic anhydrase inhibitor and/or the urease inhibitor is administered at a non-cytotoxic dose to the microorganism, thereby increasing sensitivity of the biofilm-producing bacteria to the anti-microbial agent.

According to some embodiments of the invention, the method comprising contacting the microorganism with an anti-microbial agent.

According to an aspect of some embodiments of the present invention there is provided a method of reducing or preventing formation of a biofilm and/or disrupting a biofilm, the method comprising contacting a biofilm-producing bacteria with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby reducing or preventing formation of the biofilm and/or disrupting the biofilm.

According to an aspect of some embodiments of the present invention there is provided a method of reducing or preventing formation of a biofilm and/or disrupting a biofilm, the method comprising contacting a biofilm-producing bacteria with a carbonic anhydrase inhibitor and/or a urease inhibitor and an antimicrobial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby reducing or preventing formation of the biofilm and/or disrupting the biofilm.

According to an aspect of some embodiments of the present invention there is provided a method of increasing sensitivity of a biofilm-producing bacteria to an anti-microbial agent, the method comprising contacting the biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby increasing sensitivity of the biofilm-producing bacteria to the anti-microbial agent.

According to an aspect of some embodiments of the present invention there is provided a method of increasing sensitivity of a biofilm-producing bacteria to an anti-microbial agent, the method comprising contacting the biofilm-producing microorganism with a carbonic anhydrase inhibitor and/or a urease inhibitor and an antimicrobial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby increasing sensitivity of the biofilm-producing bacteria to the anti-microbial agent.

According to some embodiments of the invention, the method being effected in-vitro or ex-vivo.

According to some embodiments of the invention, the method begin effected in-vivo.

According to an aspect of some embodiments of the present invention there is provided a method of treating a medical condition in which reducing or preventing formation of a biofilm and/or disrupting a biofilm is beneficial in a subject in need thereof, the method comprising administering to the subject a therapeutically effective amount of at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when the agent is the carbonic anhydrase inhibitor or the urease inhibitor the at least 1 agent is at least 2 agents,

thereby treating the medical condition in the subject.

According to an aspect of some embodiments of the present invention there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and a urease inhibitor, thereby treating the medical condition in the subject.

According to an aspect of some embodiments of the present invention there is provided at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

for use in the treatment of a medical condition in which reducing or preventing formation of a biofilm and/or disrupting a biofilm is beneficial, wherein when the agent is the carbonic anhydrase inhibitor or the urease inhibitor the at least 1 agent is at least 2 agents.

According to an aspect of some embodiments of the present invention there is provided a carbonic anhydrase inhibitor and a urease inhibitor for use in the treatment of a medical condition in which reducing or preventing formation of a biofilm and/or disrupting a biofilm is beneficial.

According to some embodiments of the invention, the carbonic anhydrase inhibitor and/or the urease inhibitor is administered at a non-cytotoxic dose to a microorganism producing the biofilm.

According to some embodiments of the invention, the agent is administered at a non-cytotoxic dose to a microorganism producing the biofilm.

According to an aspect of some embodiments of the present invention there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and/or a urease inhibitor, wherein the carbonic anhydrase inhibitor and/or the urease inhibitor is administered at a non-cytotoxic dose to a microorganism producing the biofilm, thereby treating the medical condition in the subject.

According to an aspect of some embodiments of the present invention there is provided a carbonic anhydrase inhibitor and/or a urease inhibitor for use in the treatment of a medical condition in which reducing or preventing formation of a biofilm and/or disrupting a biofilm is beneficial, wherein the carbonic anhydrase inhibitor and/or the urease inhibitor is administered at a non-cytotoxic dose to a microorganism producing the biofilm.

According to some embodiments of the invention, the method comprising administering to the subject an additional therapy for the medical condition.

According to some embodiments of the invention, the inhibitors or the agent further comprising an additional therapy for the medical condition.

According to some embodiments of the invention, the method comprising administering to the subject an anti-microbial agent.

According to some embodiments of the invention, the inhibitors or the agent further comprising an anti-microbial agent. According to some embodiments of the invention, the anti-microbial agent is selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an aspect of some embodiments of the present invention there is provided a method of treating a medical condition in which reducing or preventing formation of a biofilm and/or disrupting a biofilm is beneficial in a subject in need thereof, the method comprising administering to the subject a therapeutically effective amount of at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby treating the medical condition in the subject.

According to an aspect of some embodiments of the present invention there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby treating the medical condition in the subject.

According to an aspect of some embodiments of the present invention there is provided at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, for use in in the treatment of a medical condition in which reducing or preventing formation of a biofilm and/or disrupting a biofilm is beneficial.

According to an aspect of some embodiments of the present invention there is provided a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, for use in in the treatment of a medical condition in which reducing or preventing formation of a biofilm and/or disrupting a biofilm is beneficial.

According to some embodiments of the invention, the medical condition is selected from the group consisting of chronic otitis media, chronic sinusitis, chronic tonsillitis, dental plaque, chronic laryngitis, endocarditis, lung infection, kidney stones, biliary tract infections, vaginosis, osteomyelitis and chronic wounds.

According to some embodiments of the invention, the medical condition is cystic fibrosis.

According to some embodiments of the invention, the medical condition is a device related infection.

According to an aspect of some embodiments of the present invention there is provided an article of manufacture comprising at least 2 agents selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator.

According to an aspect of some embodiments of the present invention there is provided an article of manufacture comprising a carbonic anhydrase inhibitor and a urease inhibitor.

According to an aspect of some embodiments of the present invention there is provided an article of manufacture comprising at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an aspect of some embodiments of the present invention there is provided an article of manufacture comprising a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an aspect of some embodiments of the present invention there is provided an article of manufacture comprising:

(a) a solid support; and

(b) at least 1 agent coating or attached to the solid support, wherein the at least 1 agent is selected from the group consisting of:
(i) a carbonic anhydrase inhibitor;
(ii) a urease inhibitor;
(iii) a Ca²⁺ ATPase inhibitor;
(iv) a tlp activator; and
(v) a myo-inositol catabolism pathway activator,

wherein when the agent is the carbonic anhydrase inhibitor or the urease inhibitor the at least 1 agent is at least 2 agents.

According to an aspect of some embodiments of the present invention there is provided an article of manufacture comprising:

(i) a solid support; and

(ii) a carbonic anhydrase inhibitor and a urease inhibitor coating or attached to the solid support.

According to some embodiments of the invention, the article of manufacture comprising an anti-microbial agent.

According to an aspect of some embodiments of the present invention there is provided an article of manufacture comprising:

(a) a solid support;

(b) at least 1 agent coating or attached to the solid support, wherein the at least 1 agent is selected from the group consisting of:
(i) a carbonic anhydrase inhibitor;
(ii) a urease inhibitor;
(iii) a Ca²⁺ ATPase inhibitor;
(iv) a tlp activator; and
(v) a myo-inositol catabolism pathway activator; and

(c) an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an aspect of some embodiments of the present invention there is provided an article of manufacture comprising:

(i) a solid support;

(ii) a carbonic anhydrase inhibitor and/or a urease inhibitor coating or attached to the solid support; and

(iii) an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an aspect of some embodiments of the present invention there is provided a method of inducing or increasing formation of a biofilm and/or biomineralization, the method comprising contacting a biofilm-producing microorganism with at least 1 agent selected from the group consisting of:

(i) a Ca²⁺ ATPase activator;

(ii) a tlp inhibitor; and

(iii) a myo-inositol catabolism pathway inhibitor,

thereby inducing or increasing formation of the biofilm and/or the biomineralization.

According to some embodiments of the invention, the method comprising contacting the microorganism with an agent selected from the group consisting of a carbonic anhydrase activator and a urease activator.

According to an aspect of some embodiments of the present invention there is provided a microorganism obtainable by the method.

According to an aspect of some embodiments of the present invention there is provided an industrial product selected from the group consisting of a water cleaning system, a bioremediation system, a microbial leaching system, a biofilm reactor, a microbial fuel cell (MFC), a construction material and a biologic glue, comprising the microorganism.

According to some embodiments of the invention, the microorganism is not pathogenic.

According to some embodiments of the invention, the biofilm is a bacterial biofilm.

According to some embodiments of the invention, the microorganism is a bacterium.

According to some embodiments of the invention, the bacterium is selected from the group consisting of Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia and Xanthomonas.

According to some embodiments of the invention, the bacterium is selected from the group consisting of Pseudomonas aeruginosa, Enterococcus faecalis, Staphylococcus aureus, Proteus mirabolis, Pathogenic Escherichia coli and Salmonella Typhimurium.

According to some embodiments of the invention, the microorganism is not Helicobacter Pylori.

According to some embodiments of the invention, the bacterium is selected from the group consisting of Bacillus simplex, Bacillus simplexmegaterium, Bacillus sp., Bacillus brevis and Bacillus licheniformis.

According to some embodiments of the invention, the urease inhibitor and/or the carbonic anhydrase inhibitor is a small molecule.

According to some embodiments of the invention, the agent is a small molecule.

According to some embodiments of the invention, the urease inhibitor is selected from the group consisting of AHA, N-(n-butyl)thiophosphoric triamid, ecabet sodium and Epiberberin.

According to some embodiments of the invention, the carbonic anhydrase inhibitor is selected from the group consisting of Acetazolamide, 5,5′-Dithiobis (2-nitrobenzoic acid, DTNB), sulfumates, sulfamides, brimonidine, N,N-diethyldithiocarbamate, phenylboronic acid and phenylarsonic acid.

According to some embodiments of the invention, the Ca²⁺ ATPase is YloB.

According to some embodiments of the invention, the Ca²⁺ ATPase inhibitor is selected from the group consisting of sodium vanadate, EGTA and 1,2-bis(o-aminophenoxy)ethane-N,N,N′,N′-tetraacetic acid (BAPTA), N,N,N′,N′-tetrakis(2-pyridylmethyl)ethane-1,2-diamine (TPEN) and phenanthroline.

According to some embodiments of the invention, the urease inhibitor and/or the carbonic anhydrase inhibitor is an antibody, a peptide or an aptamer.

According to some embodiments of the invention, the agent is an antibody, a peptide or an aptamer.

According to some embodiments of the invention, the myo-inositol catabolism pathway activator increases expression and/or activity of the iol regulon.

According to some embodiments of the invention, the myo-inositol catabolism pathway activator inhibits expression and/or activity of iol R.

According to some embodiments of the invention, the myo-inositol catabolism pathway activator is myo-inositol or inositol or a catabolic product thereof.

According to some embodiments of the invention, the myo-inositol catabolism pathway inhibitor inhibits expression and/or activity of the iol regulon.

According to some embodiments of the invention, the myo-inositol catabolism pathway inhibitor increases expression and/or activity of iol R.

According to some embodiments of the invention, the at least 1 agent is at least 2 agents.

According to an aspect of some embodiments of the present invention there is provided a method of predicting sensitivity of a biofilm to an anti-microbial agent, the method comprising determining a concentration and/or thickness of a layer of calcium carbonate within the biofilm, wherein a concentration of the calcium carbonate and/or a thickness of a layer of the calcium carbonate above a predetermined threshold indicates the biofilm is resistant to the anti-microbial agent. According to some embodiments of the invention, the determining is effected in-vivo in a subject diagnosed with a biofilm infection.

According to some embodiments of the invention, the determining is effected in-vitro or ex-vivo on a biofilm sample obtained from a subject diagnosed with a biofilm infection.

According to some embodiments of the invention, the determining is effected by micro-CT.

According to some embodiments of the invention, the determining is effected by a 3D analysis.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E demonstrate calcium-rich structures in B. subtilis and M. smegmatis colonies grown on 1.5% B4 agar, supplemented with Ca acetate. FIG. 1A demonstrates micro-CT analysis of the samples. Left panel—3D reconstruction; Middle panel—segmentation of the reconstructed volume, red indicates the densest mineral; and Right panel—transversal slices. Scale bar: 200 μm. FIG. 1B is a graph demonstrating the relative volume of the mineral layer out of the total B. subtilis colony volume as determined by μCT X-ray. Shown are averages ±standard deviation of three independent experiments. FIG. 1C is a bar demonstrating TGA analysis of calcium minerals in B. subtilis colonies. Shown are averages ±standard deviation of three independent experiments. FIG. 1D is a graph demonstrating the estimated thickness of the mineral layer in B. subtilis colony as determined by the parallel plate model. Shown are averages ±standard deviation of three independent experiments. FIG. 1E is a graph demonstrating that calcium carbonate is the mineral accumulating within B. subtilis colony biofilms as determined by FTIR analysis.

FIGS. 2A-C demonstrate that diffusion through B. subtilis and M. smegmatis biofilm colonies is limited by calcium-dependent barriers. B. subtilis and M. smegmatis colonies grown on 1.5% B4 agar, supplemented with Ca acetate. FIG. 2A shows images of fluorescein isothiocyanate (FITC) diffusion in B. subtilis biofilm colonies. Upper left—bright field, lower left—GFP filer, right panel—enlargement of the lower left image. Scale bars represent 2 mm. FIG. 2B shows images of cross-sections of colonies taken 4 hours following addition of FITC dye. Scale bars represent 0.5 mm. FIG. 2C shows histograms of cells stained with CalceinAM and analyzed by FACS. Shown are two independent repeats per treatment.

FIGS. 2D-F demonstrate calcium carbonate structures in P. aeruginosa colonies grown on 1.5% B4 agar, supplemented with Ca acetate. FIG. 2D shows phase and micro-CT X-ray images of structured biofilms. FIG. 2E shows images of phase and fluorescent calcium immunostatining of an intact colony biofilm using a calcein derivate that cannot penetrate the cell membrane. FIG. 2F is a graph demonstrating that calcium carbonate is the mineral accumulating within the colony biofilm as determined by FTIR analysis.

FIGS. 3A-E demonstrate that urease inhibition with acetohydroxamic acid (AHA) inhibits growth, biofilm development and biomineralization of B. subtilis and M. smegmatis colonies. B. subtilis and M. smegmatis colonies grown on 1.5% B4 agar, supplemented with Ca acetate. FIG. 3A is a schematic representation of the biomineralization reactions leading to bicarbonate production. FIG. 3B shows images of B. subtilis colonies grown in a medium supplemented with 0.25% Ca and treated with the indicated doses of acetohydroxamic acid (AHA). Scale bars represent 2 mm. FIG. 3C is a graph demonstrating planktonic growth of B. subtilis (upper graph) and M. smegmatis (lower graph) in B4 medium supplemented with Ca and AHA at the indicated concentrations. FIG. 3D shows images of M. smegmatis colonies grown in a medium supplemented with 0.025% Ca and treated with AHA at the indicated concentrations. DMSO served as a positive control and a medium not supplemented with Ca served as a negative control. Scale bars represent 2 mm. FIG. 3E shows images of day 3 B. subtilis colonies and cross-sections of colonies 4 hours following addition of FITC dye. Colonies were grown in a medium supplemented with 0.025% Ca and treated with 10 mg/ml AHA. DMSO served as a positive control and a medium not supplemented with Ca served as a negative control. Scale bars represent 2 mm in the upper panels; and 0.5 mm in the lower panels.

FIGS. 4A-B demonstrate that AHA inhibits growth, biofilm development and biomineralization of B. subtilis and M. smegmatis colonies in a pellicle biofilm model. B. subtilis (FIG. 4A) and M. smegmatis (FIG. 4B) cells were grown in liquid B4 supplemented with 0.25% and 0.025% calcium acetate, respectively, and treated with AHA at the indicated concentrations. DMSO served as a positive control and a medium not supplemented with Ca served as a negative control. The images of the top view of the wells were taken following robust pellicles formed in the DMSO control—at 3 days for B. subtilis and at 4 days for M. smegmatis.

FIG. 5 demonstrates that inhibition of urease with AHA decreases non-soluble mineral production in B. subtilis colonies in a pellicle biofilm model. B. subtilis cells were grown in liquid B4 supplemented with 0.25% calcium acetate and treated with AHA at the indicated concentrations. DMSO served as a positive control and a medium not supplemented with Ca served as a negative control. The images of the top view of the wells were taken following 6 days; and the weight of the mineral was determined following removal of all organic material by bleaching.

FIG. 6 is a bar graph demonstrating the effects of inhibitors of carbonic anhydrase and urease on submerged biofilm formation as determined by Crystal Violet Staining.

FIG. 7 is a bar graph demonstrating the effects of combined treatment with carbonic anhydrase and urease inhibitors on the survival of the P. aeruginosa biofilm colony cells following exposure to ciprofloxacin.

FIG. 8 is a bar graph demonstrating the effects of inhibitors of Ca2+-ATPase, carbonic anhydrase and urease on P. aeruginosa pre-existing biofilm, as determined by Crystal Violet Staining. P. aeruginosa biofilms were grown in multi-well 24 wells plates in TSB for 12 hours. Following, biofilms were treated either in PBS or PBS applied with the indicated concentrations of sodium vanadate, DTNB or AHA for 6 hours. Results are shown as intensity of crystal violet staining (OD595) compared with the initial absorbance prior to treatment.

FIG. 9 is a bar graph demonstrating the effects of treatment with carbonic anhydrase and urease inhibitors on the survival of the P. aeruginosa pre-existing biofilm following exposure to ciprofloxacin (CIP) or gentamicin (GM). P. aeruginosa biofilms were grown in multi-well 24 wells plates in TSB for 12 hours. Following, biofilms were treated either in PBS or PBS applied with the indicated concentrations of DTNB, AHA, CIP and/or GM for 6 hours. Following treatment, the biofilms were mildly sonicated and serially diluted to assess the number of CFU within each group.

FIGS. 10A-D demonstrates that calcification promotes persistent infections and depends on the same metabolic pathways. FIG. 10A is a scanning Electron Microscopy (SEM) image of biofilms of partially bleached sputum sample from P. aeruginosa positive CF patient A4, containing bleach-resistant mineralized tissue. FIG. 10B is a SEM image of biofilms of partially bleached sputum sample from P. aeruginosa positive CF patient A62 (left), accompanied by an image taken with the backscattering mode (right), containing bleach-resistant mineralized tissue. FIG. 10C shows a fully bleached sputum sample of P. aeruginosa positive CF patients. Patient 463125: calcite crystals demonstrated by FTIR analysis (left), and patient 463123: calcite crystals visualized by SEM (right). FIG. 10D shows the Ex-vivo system to study P. aeruginosa lung infection. P. aeruginosa strain PA14 expressing GFP (green) was used to infect a lung tissue (nucleoli of lung cells were stained with DAPI—blue). Lung tissue was harvested from one month old mice and incubated in DMEM 5% FCS at 37° C. for 2 days, in the presence of P. aeruginosa, sectioned and visualized. Shown are histologic images (magnification ×40) demonstrating that blocking biomineralization by the urease inhibitor AHA prevents tissue damage in an ex vivo system. Lungs were harvested from one month old mice and tissue was incubated in DMEM 5% FCS, either with or without AHA, as indicated. To each sample, either P. aeruginosa or DMEM (control) was added. Samples were incubated at 37° C. for 2 days, sectioned, stained with Hematoxylin/eosin stain and visualized.

FIG. 11 shows histologic images (magnification ×20) demonstrating that blocking biomineralization by the carbonic anhydrase inhibitor DTNB prevents tissue damage in an ex vivo system. Lungs were harvested from one month old mice and tissue was incubated in DMEM 5% FCS, either with or without DTNB, as indicated. To each sample, either P. aeruginosa or DMEM (control) was added. Samples were incubated at 37° C. for 2 days, sectioned, stained with Hematoxylin/eosin stain and visualized.

FIGS. 12A-F demonstrate the role of the iol regulon, YloB and Tlp in regulating calcification and biofilm formation. FIG. 12A shows DAVID analysis of the transcriptome highlighted the myo-inositol synthesis pathway. Upregulated genes are indicated in red.

FIG. 12B is a graph demonstrating average fold change at days 1, 2 and 3 of indicated genes. FIG. 12C shows images of biofilm colonies of wild-type B. subtilis (NCIB 3610) strain and its mutant derivatives. Colonies were grown on solid B4 biofilm-inducing medium, with or without calcium as indicated, and images were taken at day 3 (D3) and day 6 (D6). FIG. 12D shows images of biofilm colonies of wild-type B. subtilis (NCIB 3610) strain and its mutant ΔyloB. Colonies were grown on B4 agar plates supplemented with calcium acetate. At day 6, the intact colonies were visualized under X-ray. FIG. 12E shows thermogravimetric analysis (TGA) of lyophilized biofilm colonies of wild-type and Δtlp and ΔiolR mutants. The range 150-1000° C. was used for calculation of the total organic matrix content. The peak at 2000° C. to 5700° C. is organic matter. The peak at 761° C. marks the decomposition of mineral. Δtlp and ΔiolR significantly differed from the wild-type in three independent experiments (P-value 0.1 and 0.025 respectively). FIG. 12F is a graph demonstrating no effect of ΔYloB, Δtlp and ΔiolR on planktonic growth. Wild type and its mutant derivatives were grown at 30° C. with shaking in liquid B4 medium with and without calcium, and growth was monitored by measuring OD600 in a microplate reader every 15 mins. Results are averages of three wells within three experiments, bars represent standard deviations.

FIG. 13 demonstrates the effects of the YloB inhibitor sodium vanadate and the urease inhibitor AHA on P. aeruginosa biofilm formation as determined by Crystal Violet Staining.

FIG. 14 demonstrates the effects of the YloB inhibitor sodium vanadate and the carbonic anhydrase inhibitor DTNB on P. aeruginosa biofilm formation as determined by Crystal Violet Staining.

### DESCRIPTION OF SPECIFIC EMBODIMENTS OF THE INVENTION

The present invention, in some embodiments thereof, relates to methods of disrupting a biofilm and/or preventing formation of same.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Formation of biofilm of bacteria and other microorganisms and resistance to anti-microbial agents, present a challenge in the battle against infections and other medical conditions associated with pathogenic microorganisms, biofouling of medical devices, particularly in internal medicinal and dentistry fields, as well as in fields such as water treatment, containment and transportation.

Whilst reducing the present invention to practice, the present inventors have now uncovered that urease inhibitors, carbonic anhydrase inhibitors, Ca²⁺ ATPase inhibitors and/or myo-inositol catabolism pathway activators inhibit formation of a biofilm, increase biofilm permeability and sensitize the bacteria to bactericides treatment, while tlp inhibitors increase the formation of biofilm.

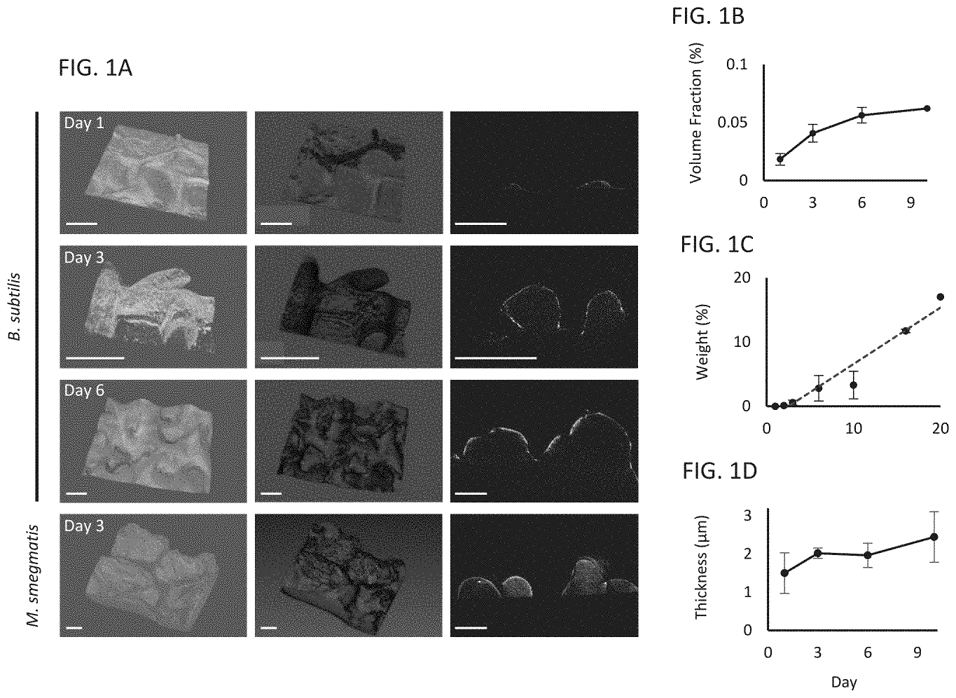

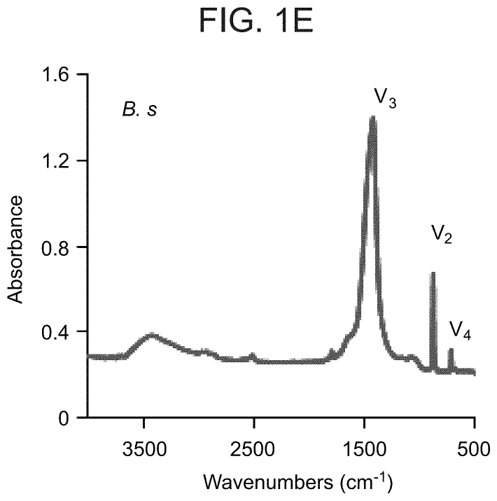

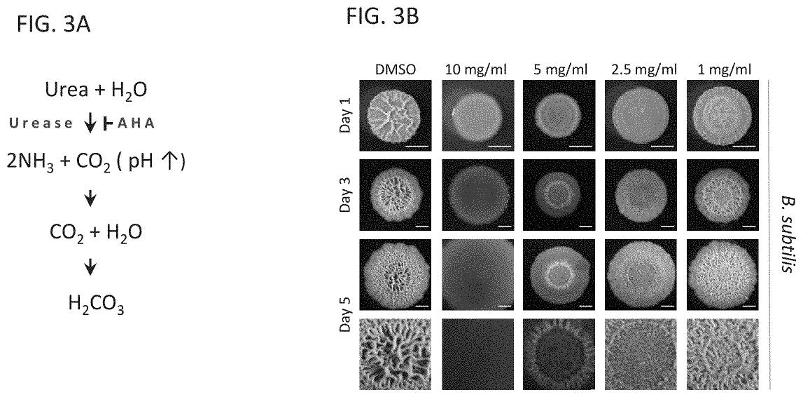

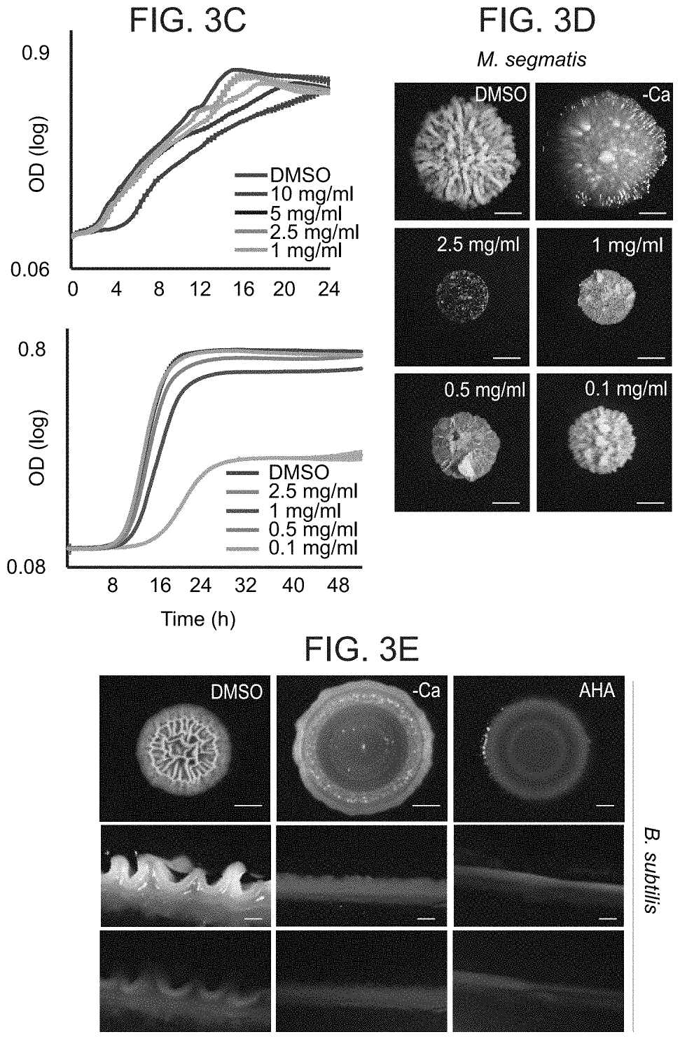

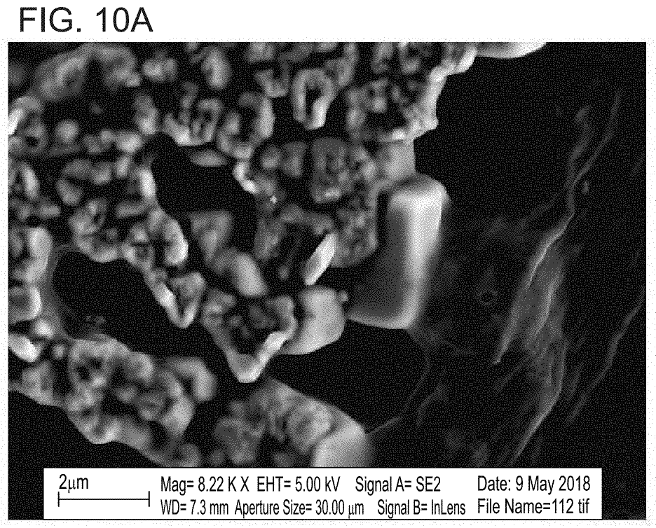

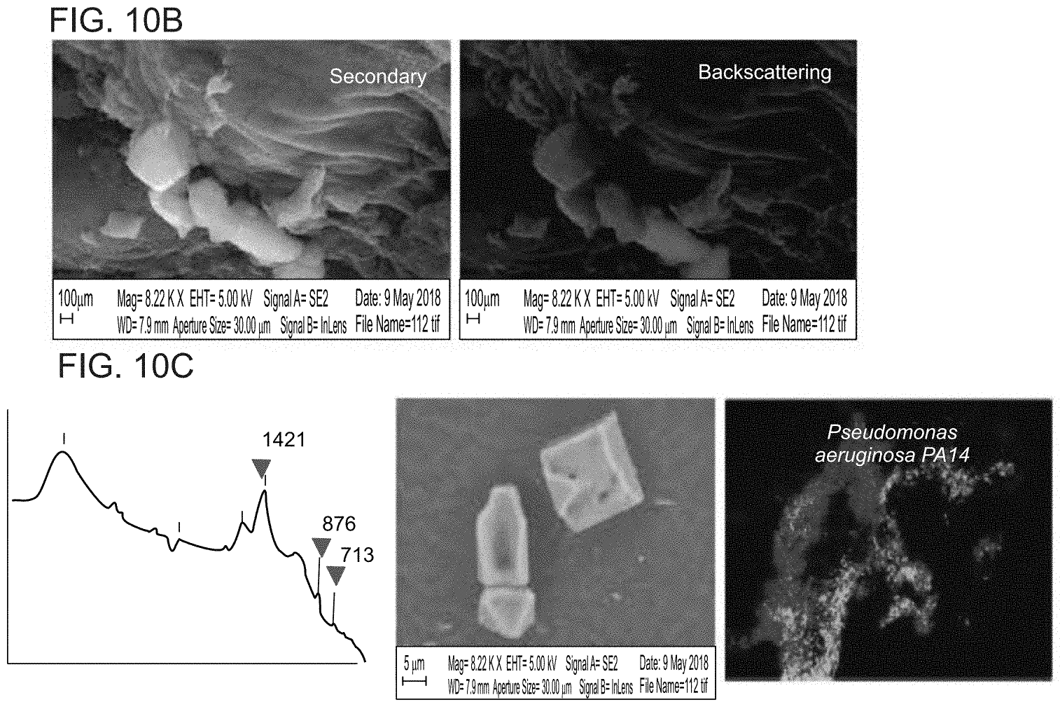

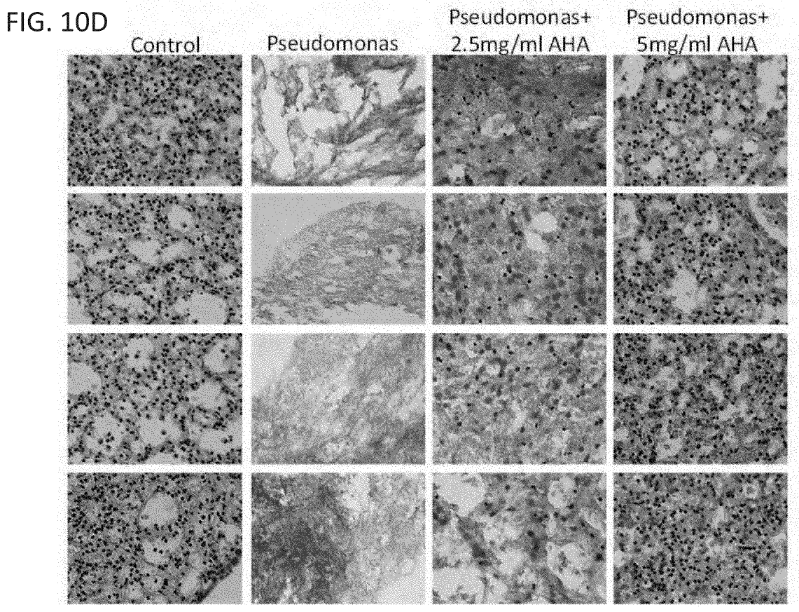

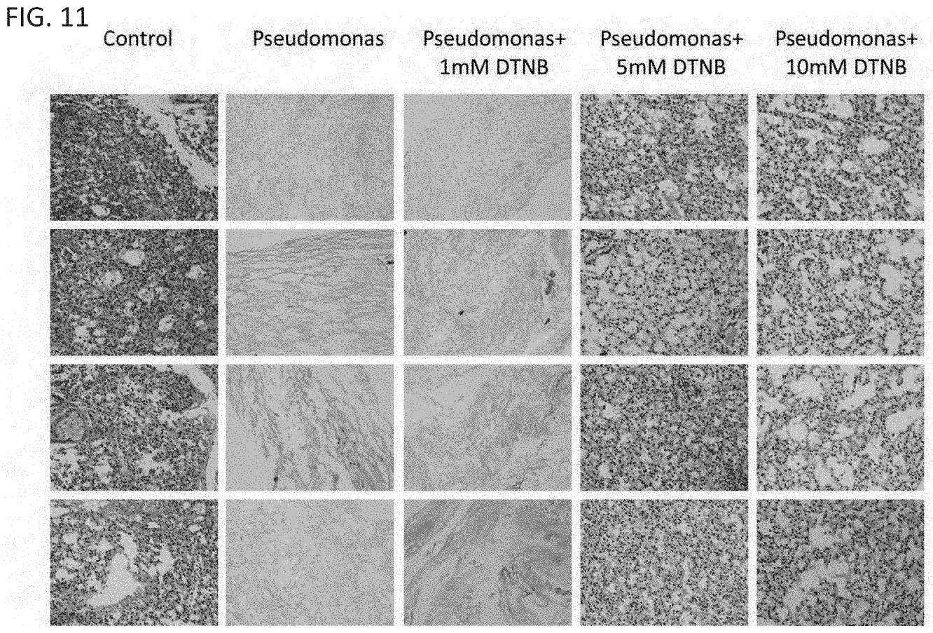

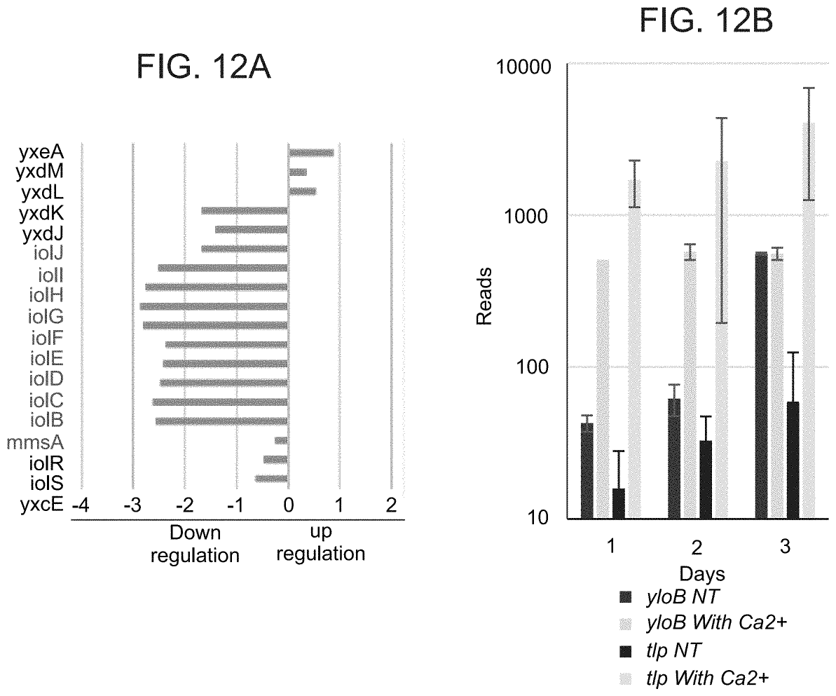

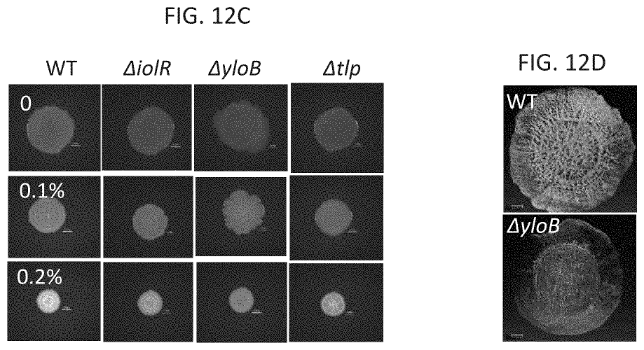

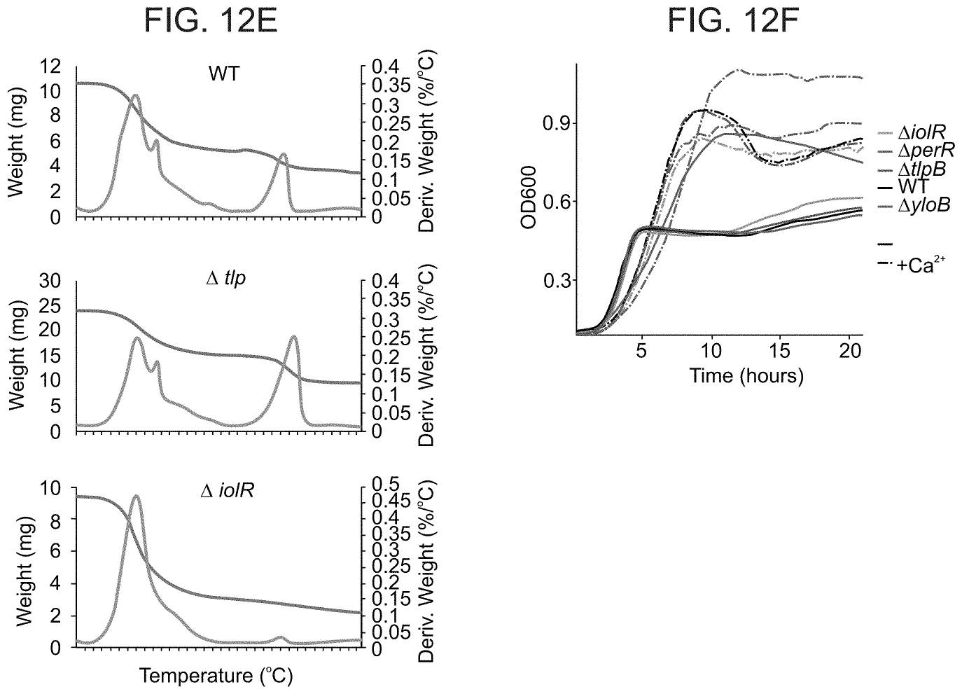

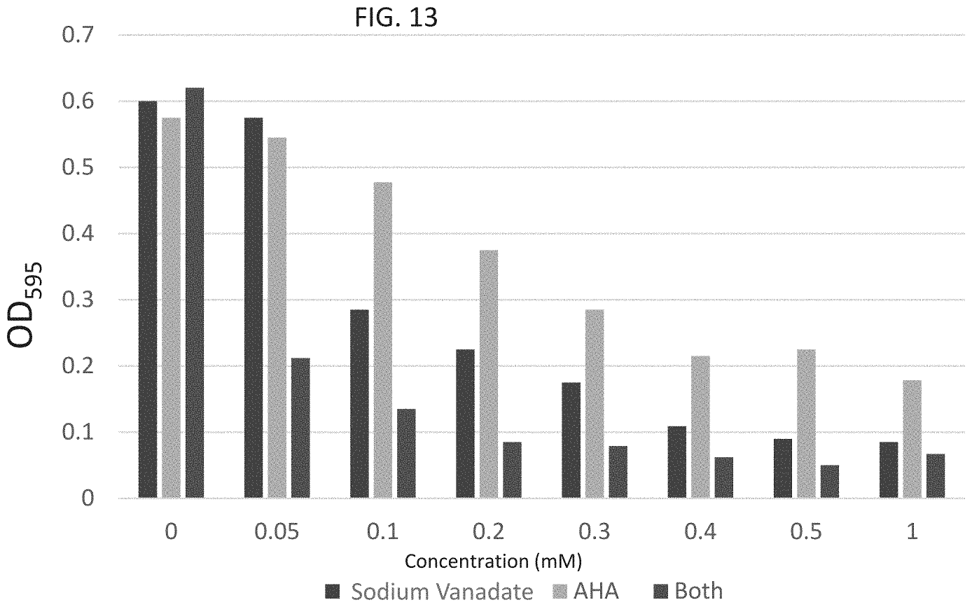

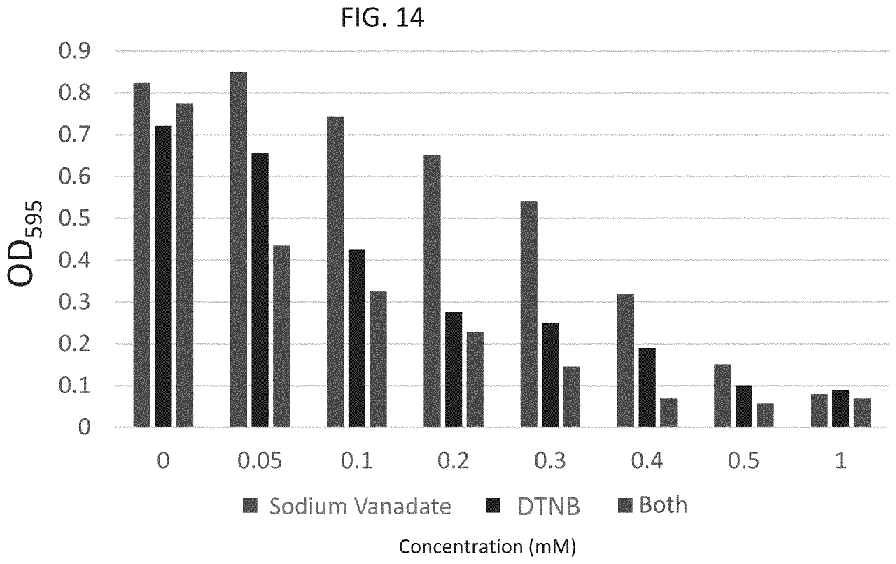

As is illustrated hereinunder and in the examples section, which follows, the present inventors have discovered that biomineralization of extracellular calcium carbonate sheets in bacterial biofilm of several distinct species appear in parallel to complex colony formation. Furthermore, these calcium carbonate sheets serve as a rigid mineral layer potentially increasing the weight bearing of the wrinkles and supporting the overall colony structure; and also as a diffusion barrier sheltering the inner cell mass of the biofilm colony (Example 1, FIGS. 1A-E, 2A-F). In addition, the present inventors show that chemical inhibition of urease (using AHA) and/or carbonic anhydrase (using DTNB) at non-bactericidal doses inhibits assembly of complex bacterial structures, prevents formation of the protective diffusion barriers, disperses pre-existing biofilm, increases biofilm permeability and sensitizes the bacteria to bactericides treatment (Example 2, FIGS. 3A-C, 4A-B and 5-9). Moreover, chemical inhibition of urease (using AHA) and/or carbonic anhydrase (using DTNB) lead to diminished P. aeruginosa lung colonization and prevented lung tissue death in an ex-vivo lung infection system (Example 3, FIGS. 10A-11). In addition, using transcriptome and mutagenesis analysis, the present inventors show that deletion of iolR (the repressor of the iol regulon), YloB (Ca2+ ATPase) or Tlp prevents biomineralization and biofilm formation (Example 4, FIGS. 12A-F). Furthermore, chemical inhibition of Ca2+ ATPase (using sodium vanadate) inhibits biofilm formation (Example 5, FIGS. 13-14).

Thus, according to a first aspect of the present invention, there is provided a method of reducing or preventing formation of a biofilm and/or disrupting a biofilm, the method comprising contacting a biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when said agent is said carbonic anhydrase inhibitor or said urease inhibitor said at least 1 agent is at least 2 agents,

thereby reducing or preventing formation of the biofilm and/or disrupting the biofilm.

According to an alternative or an additional aspect of the present invention, there is provided a method of reducing or preventing formation of a biofilm and/or disrupting a biofilm, the method comprising contacting a biofilm-producing microorganism with a carbonic anhydrase inhibitor and a urease inhibitor, thereby reducing or preventing formation of the biofilm and/or disrupting the biofilm.

According to an alternative or an additional aspect of the present invention, there is provided a method of reducing or preventing formation of a biofilm and/or disrupting a biofilm, the method comprising contacting a biofilm-producing bacteria with a carbonic anhydrase inhibitor and/or a urease inhibitor, wherein said carbonic anhydrase inhibitor and/or said urease inhibitor is administered at a non-cytotoxic dose to said microorganism, thereby reducing or preventing formation of the biofilm and/or disrupting the biofilm.

According to an alternative or an additional aspect of the present invention, there is provided a method of reducing or preventing formation of a biofilm and/or disrupting a biofilm, the method comprising contacting a biofilm-producing bacteria with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby reducing or preventing formation of the biofilm and/or disrupting the biofilm.

According to an alternative or an additional aspect of the present invention, there is provided a method of reducing or preventing formation of a biofilm and/or disrupting a biofilm, the method comprising contacting a biofilm-producing bacteria with a carbonic anhydrase inhibitor and/or a urease inhibitor and an antimicrobial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby reducing or preventing formation of the biofilm and/or disrupting the biofilm.

As used herein, the term “carbonic anhydrase”, EC No. 4.2.1.1, refers to an enzyme which catalyzes interconversion of carbon dioxide and water to bicarbonate and protons. Methods of determining the catalytic activity of carbonic anhydrase are well known in the art and include, but are not limited to, manometric methods such as described e.g. in MELDRUM and ROUGHTON, 1933; KIESE and HASTINGS, 1940; and VAN GOOR, 1948; colorimetric methods such as described e.g. in PHILPOT and PHILPOT, 1936, NYMAN, 1963); electrometric methods such as described e.g. in WILBUR and ANDERSON, 1948; Raymond P. Henry, The Carbonic Anhydrases pp 119-125; micro method such as described e.g. in MAREN, 1960; electrometric and titrimetric methods such as described e.g. in WORTHINGTON, 1988); and spectrophotometric methods such as described e.g. in Prasanta K.Datta et al. Archives of Biochemistry and Biophysics, 1959, 79: 136-145; the contents of which are fully incorporated herein by reference. Kits for assaying carbonic anhydrase activity are also commercially available from e.g. BioVision.

According to a specific embodiment, the carbonic anhydrase is present in biofilm of biofilm-producing microorganisms.

As used herein, the phrase “carbonic anhydrase inhibitor” refers to an agent capable of binding carbonic anhydrase or a polynucleotide encoding same and inhibiting its expression or catalytic activity.

According to specific embodiments, the carbonic anhydrase inhibitor inhibits carbonic anhydrase activity.

As used herein, the term “urease”, EC No. 3.5.1.5, refers to an enzyme, which catalyzes the hydrolysis of urea into carbon dioxide and ammonia. Methods of determining the catalytic activity of carbonic anhydrase are well known in the art and include, but are not limited to, manometric methods, titrimetric methods, colorimetric methods, electrochemical methods and Spectrophotometric Methods such as described e.g. in Donald D. Van Slyke and Reginald M. J. Biol. Chem. 1944, 154:623-642; and Joseph G. Montalvo Anal. Chem., 1969, 41 (14): 2093-2094, the contents of which are fully incorporated herein by reference. A spot test for urease activity in gram positive bacteria has also been described by S. M. HUSSAIN QADRI et al., JOURNAL OF CLINICAL MICROBIOLOGY, 1984, 1198-1199. Kits for assaying urease activity are also commercially available from e.g. Abnova, Sigma and Abcam.

According to a specific embodiment, the urease is present in biofilm of biofilm-producing microorganisms.

As used herein, the phrase “urease inhibitor” refers to an agent capable of binding urease or a polynucleotide encoding same and inhibiting its expression or catalytic activity.

According to specific embodiments, the urease inhibitor inhibits urease activity.

As used herein, the term “Ca²⁺ ATPase”, EC No. 7.2.2.10, refers to an enzyme, which catalyzes the hydrolysis of ATP coupled with the transport of calcium.

According to specific embodiments, the Ca²⁺ ATPase us a plasma membrane Ca²⁺ ATPase.

Methods of determining the catalytic activity of Ca²⁺ ATPase are well known in the art and include, but are not limited to, determination of the released inorganic phosphate optionally with the addition of the transported Ca2+ ions using the methods described e.g. in Fiske & Subbarow (1925), Ames (1962), Lanzetta et al. (1979) and Chan et al. (1986), the contents of which are fully incorporated herein by reference. Kits for assaying Ca²⁺ ATPase activity are also commercially available from e.g. Innova Biosciences.

According to specific embodiments, the Ca²⁺ ATPase is YloB (encoded by Gene ID: 936954).

According to specific embodiments, the YloB is the Bacillus subtilis YloB, such as provided in UniProt No 034431.

According to specific embodiments, the YloB amino acid sequence comprises SEQ ID NO: 13.

According to specific embodiments, the YloB amino acid sequence consists of SEQ ID NO: 13.

The term “YloB” also encompasses functional homologues (naturally occurring or synthetically/recombinantly produced), which exhibit the desired activity (i.e., Ca²⁺ ATPase). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 13; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

Sequence identity or homology can be determined using any protein or nucleic acid sequence alignment algorithm such as Blast, ClustalW, and MUSCLE.

The homolog may also refer to an ortholog, a deletion, insertion, or substitution variant, including an amino acid substitution.

According to a specific embodiment, the Ca²⁺ ATPase (e.g. YloB) is present in biofilm of biofilm-producing microorganisms.

As used herein, the phrase “Ca²⁺ ATPase inhibitor” refers to an agent capable of binding Ca²⁺ ATPase or a polynucleotide encoding same and inhibiting its expression or catalytic activity.

According to specific embodiments, the Ca²⁺ ATPase inhibitor inhibits Ca²⁺ ATPase activity.

As used herein, the term “tlp” refers to a charged thioredoxin-like protein encoded by the sspT gene (Gene ID: 938091). According to specific embodiments, tlp activity is reduction of oxidized cysteine residues and the cleavage of disulfide bonds. Methods of determining activity of tlp are well known in the art and include, but are not limited to the insulin reduction assay. Kits for assaying tlp activity are also commercially available from e.g. Cayman Chmical.

According to specific embodiments, the tlp is the Bacillus subtilis tlp, such as provided in UniProt No Q45060.

According to specific embodiments, the tlp amino acid sequence comprises SEQ ID NO: 14.

According to specific embodiments, the tlp amino acid sequence consists of SEQ ID NO: 14.

The term “tlp” also encompasses functional homologues (naturally occurring or synthetically/recombinantly produced), which exhibit the desired activity (i.e., reduction of oxidized cysteine residues and the cleavage of disulfide bonds). Such homologues can be, for example, at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical or homologous to the polypeptide SEQ ID No: 14; or at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the polynucleotide sequence encoding same.

According to a specific embodiment, the tlp is present in biofilm of biofilm-producing microorganisms.

As used herein, the phrase “tlp activator” refers to an agent capable of increasing tlp expression or activity.

According to specific embodiments, the tlp activator increases tlp expression.

According to specific embodiments, the tlp activator increased tlp activity.

As used herein, the phrase “myo-inositol catabolism pathway” refers to any enzyme, regulator, substrate or catabolic product being part of the multiple stepwise conversion of myo-inositol (CAS Number 87-89-8) to acetyl-CoA (CAS Number 72-89-9) and CO2.

The components involved in the myo-inositol catabolism pathway are known to the skilled in the art and include, but not limited to:
the enzymes myo-inositol dehydrogenase, 2KMI dehydrogenase, THcHDO hydrolase, 5DG isomerase, DKG kinase, iolI aldolase, MSA dehydrogenase;
the compounds myo-inositol (MI), inositol, 2KMI, 3D-(3,4/5)trihydroxycyclohexane-1,2-dione (THcHDO), 4,5-deoxy-D-glucuronic acid (5DG); 2-deoxy-5-keto-D-gluconic acid (DKG); DKGP; dihydroxyacetone phosphate (DHAP); malonic semialdehyde (MSA); acetyl coenzyme A (acetyl-CoA).
the genes and polynucleotides encoding the enzymes (typically encoded by the iol operon or regulun) iolG, iolE, iolD, iolB, iolC, iolJ, iolA;
IolR (gene ID 937635), the repressor controlling transcription of the iol operon (iolABCDEFGHIJ).

Methods of determining myo-inositol catabolism are well known in the art and are described for example in Yoshida et al. (THE JOURNAL OF BIOLOGICAL CHEMISTRY VOL. 283, NO. 16, pp. 10415-10424, 2008) the contents of which are fully incorporated herein by reference.

As used herein, the phrase “myo-inositol catabolism pathway activator” refers to an agent capable of increasing myo-inositol catabolism by affecting expression, activity and/or an amount of any of the components involved in myo-inositol catabolism.

According to specific embodiments, the myo-inositol catabolism pathway activator increases expression of an enzyme involved in myo-inositol catabolism.

According to specific embodiments, the myo-inositol catabolism pathway activator increases expression and/or activity of the iol regulon.

According to specific embodiments, the myo-inositol catabolism pathway activator inhibits expression and/or activity of iolR.

According to specific embodiments, the myo-inositol catabolism pathway activator is myo-inositol or inositol or a catabolic product thereof.

According to specific embodiments, the myo-inositol catabolism pathway activator is myo-inositol.

Myo-inositol, inositol and the catabolic products thereof can be commercially available from e.g. Sigma.

According to specific embodiments, the inhibition is at least 1.5 fold, at least 2 fold, at least 3 fold, at least 5 fold, at least 10 fold, or at least 20 fold as compared to same in the absence of the inhibitor, as may be determined by e.g. any of the methods described hereinabove.

According to other specific embodiments the inhibition is by at least 5%, by at least a 10%, at least 20%, at least 30%, at least 40%, at least 50%, at least 60%, at least 70%, at least 80%, at least 90%, at least 95%, at least 99% or 100% as compared to same in the absence of the inhibitor, as may be determined by e.g. any of the methods described hereinabove.

According to specific embodiments, the activation or increase is at least 1.5 fold, at least 2 fold, at least 3 fold, at least 5 fold, at least 10 fold, or at least 20 fold as compared to same in the absence of the activator, as may be determined by e.g. any of the methods described hereinabove.

According to other specific embodiments the activation or increase is by at least 5%, by at least a 10%, at least 20%, at least 30%, at least 40%, at least 50%, at least 60%, at least 70%, at least 80%, at least 90%, at least 95%, at least 99% or 100% as compared to same in the absence of the activator, as may be determined by e.g. any of the methods described hereinabove.

Specific embodiments of the present invention comprise a single agent selected from a carbonic anhydrase inhibitor; a urease inhibitor; a Ca²⁺ ATPase inhibitor; a tlp activator; and a myo-inositol catabolism pathway activator. Specific embodiments of the present invention comprise a carbonic anhydrase inhibitor (i.e. not in combination with a urease inhibitor).

Specific embodiment of the present invention comprise a urease inhibitor (i.e. not in combination with a carbonic anhydrase inhibitor).

Other specific embodiments of the present invention comprise at least 2, at least 3, at least 4 or 5 agents selected from (i) a carbonic anhydrase inhibitor; (ii) a urease inhibitor; (iii) a Ca²⁺ ATPase inhibitor; (iv) a tlp activator; and (v) a myo-inositol catabolism pathway activator.

Specific embodiments of the present invention comprise at least 2 agents selected from (i) a carbonic anhydrase inhibitor; (ii) a urease inhibitor; (iii) a Ca²⁺ ATPase inhibitor; (iv) a tlp activator; and (v) a myo-inositol catabolism pathway activator.

Thus, specific embodiments of the present invention comprise (i)+(ii), (i)+(iii), (i)+(iv), (i)+(v), (ii)+(iii), (ii)+(iv), (ii)+(v), (iii)+(iv), (iii)+(v), (iv)+(v). (i)+(ii)+(iii), (i)+(ii)+(iv), (i)+(ii)+(iv), (i)+(iii)+(iv), (i)+(iii)+(v), (i)+(iv)+(v), (ii)+(iii)+(iv), (ii)+(iii)+(v), (ii)+(iv)+(v), (iii)+(iv)+(v), (i)+(ii)+(iii)+(iv), (i)+(ii)+(iii)+(v), (i)+(iii)+(iv)+(v), (ii)+(iii)+(iv)+(v), (i)+(ii)+(iii)+(iv)+(v), each possibility represents a separate embodiment of the present invention.

Specific embodiments of the present invention comprise a carbonic anhydrase inhibitor and a Ca²⁺ ATPase inhibitor.

Specific embodiments of the present invention comprise a urease inhibitor and a

Ca²⁺ ATPase inhibitor.

Other specific embodiments of the present invention comprise a carbonic anhydrase inhibitor and a urease inhibitor.

According to specific embodiments, a combination of the agents disclosed herein has at least an additive effect (e.g. reducing or preventing formation of a biofilm, disrupting a biofilm, increasing sensitivity of a biofilm-producing bacteria to an anti-microbial agent, treating a medical condition in which reducing or preventing formation of a biofilm and/or disrupting a biofilm is beneficial) compared to each of the agents when used alone.

According to a specific embodiment, the combination of agents has a synergistic effect.

According to specific embodiments, a combination of a carbonic anhydrase inhibitor and a urease inhibitor has at least an additive effect (e.g. reducing or preventing formation of a biofilm, disrupting a biofilm, increasing sensitivity of a biofilm-producing bacteria to an anti-microbial agent, treating a medical condition in which reducing or preventing formation of a biofilm and/or disrupting a biofilm is beneficial) compared to each of the inhibitors when used alone.

According to a specific embodiment, the combination of a carbonic anhydrase inhibitor and a urease inhibitor has a synergistic effect.

When using a combination of agents (e.g. carbonic anhydrase inhibitor and a urease inhibitor) in any of the methods and uses described herein, the agents can be contacted with the microorganism or otherwise administered to the subject, concomitantly, concurrently, simultaneously, consecutively or sequentially with one another.

Inhibiting any of the targets disclosed herein (e.g. carbonic anhydrase, urease, Ca²⁺ ATPase) can be effected on the genomic and/or the transcript level using a variety of molecules which interfere with transcription and/or translation (e.g., RNA silencing agents), or on the protein level using e.g., small molecules, antibodies, aptamers, inhibitory peptides, enzymes that cleave the polypeptide and the like.

According to specific embodiments, the inhibitor is a polynucleotide.

According to specific embodiments the inhibitor is a nucleic acid suitable for silencing expression.

As used, herein the phrase “nucleic acid suitable for silencing expression” refers to regulatory mechanisms mediated by nucleic acid molecules which result in the inhibition or “silencing” of the expression of a corresponding protein-coding gene. Numerous methods are known in the art for gene silencing in prokaryotes, examples include but are not limited to U.S. Patent Application 20040053289, which teaches the use of si hybrids to down-regulate prokaryotic genes, and U.S. Patent Application PCT/US09/69258, which teaches the use of CRISPR to downregulate prokaryotic genes.

Alternatively, according to specific embodiments, the inhibition can be carried out at the protein level, which interferes with the enzyme activity. Non-limiting examples of such inhibitors include small molecules, antibodies, inhibitory peptides, aptamers and the like.

According to specific embodiments, the inhibitor is a small molecule.

Non-limiting examples of small molecule inhibitors of carbonic anhydrase include Acetazolamide (Diamox), 5,5′-Dithiobis (2-nitrobenzoic acid, DTNB, also known as DNDB), sulfumates, sulfamides, brimonidine, N,N-diethyldithiocarbamate, phenylboronic acid, phenylarsonic acid and analogs or derivatives thereof.

According to specific embodiments, the carbonic anhydrase inhibitor is selected from the group consisting of Acetazolamide, 5,5′-Dithiobis (2-nitrobenzoic acid, DTNB), sulfumates, sulfamides, brimonidine, N,N-diethyldithiocarbamate, phenylboronic acid and phenylarsonic acid.

According to specific embodiments, the carbonic anhydrase inhibitor is Acetazolamide or an analog or derivative thereof.

According to specific embodiments, the carbonic anhydrase inhibitor is Acetazolamide.

According to specific embodiments, the carbonic anhydrase inhibitor is 5,5′-Dithiobis (2-nitrobenzoic acid, DTNB) or an analog or derivative thereof.

According to specific embodiments, the carbonic anhydrase inhibitor is 5,5′-Dithiobis (2-nitrobenzoic acid, DTNB).

According to specific embodiments, the carbonic anhydrase inhibitor is not a phenylboronic acid or a phenylarsonic acid.

According to specific embodiments, the carbonic anhydrase inhibitor is not 4-fluorophenylboronic acid.

According to specific embodiments, the carbonic anhydrase inhibitor is not a beta lactam inhibitor.

Non-limiting examples of small molecule inhibitors of urease include acetohydroxamic acid (AHA), N-(n-butyl)thiophosphoric triamid, ecabet sodium, Epiberberin and analogs or derivatives thereof.

According to specific embodiments, the urease inhibitor is selected from the group consisting of AHA, N-(n-butyl)thiophosphoric triamid, ecabet sodium and Epiberberin.

According to specific embodiments, the urease inhibitor is AHA or an analog or derivative thereof.

According to specific embodiments, the urease inhibitor is AHA.

Non-limiting examples of Ca²⁺ ATPase inhibitors include sodium vanadate, EGTA and 1,2-bis(o-aminophenoxy)ethane-N,N,N′,N′-tetraacetic acid (BAPTA), N,N,AP,N1-tetrakis(2-pyridylmethyl)ethane-1,2-diamine (TPEN), phenanthroline and analogs or derivatives thereof.

According to specific embodiments, the Ca²⁺ ATPase inhibitor is selected from the group consisting of sodium vanadate, EGTA and 1,2-bis(o-aminophenoxy)ethane-N,N,N′,N′-tetraacetic acid (BAPTA), N,N,N′,N′-tetrakis(2-pyridylmethyl)ethane-1,2-diamine (TPEN) and phenanthroline.

According to specific embodiments, the Ca²⁺ ATPase inhibitor is sodium vanadate (CAS No. 13718-26-8).

According to specific embodiments, the inhibitor is an antibody, a peptide or an aptamer.

According to specific embodiments inhibitor is an antibody capable of specifically binding the target disclosed herein (e.g. carbonic anhydrase, urease or Ca²⁺ ATPase). Preferably, the antibody specifically binds at least one epitope of the target (e.g. a carbonic anhydrase, urease or Ca²⁺ ATPase).

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen.

As some of the targets are localized intracellularly, according to specific embodiments, the antibody is an intracellular antibody.

For example, as carbonic anhydrase and urease are localized intracellularly, an antibody or antibody fragment capable of specifically binding carbonic anhydrase or urease is typically an intracellular antibody.

Methods of producing polyclonal and monoclonal antibodies, human and humanized antibodies, as well as fragments thereof are well known in the art (See for example, Harlow and Lane, Antibodies: A Laboratory Manual, Cold Spring Harbor

Laboratory, New York, 1988, incorporated herein by reference).

Another inhibitor which can be used along with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Another inhibitor would be any molecule (e.g. small molecule, peptide) which binds to and/or cleaves the target (e.g. carbonic anhydrase, urease or Ca²⁺ ATPase). Alternatively or additionally, an inhibitor may be any molecule which interferes with the target (e.g carbonic anhydrase, urease or Ca²⁺ ATPase) function (e.g., catalytic or substrate binding).

It will be appreciated that a non-functional analogue of at least a catalytic or binding portion of the target disclosed herein (e.g. carbonic anhydrase, urease, Ca²⁺

ATPase) can be also used as an inhibitor.

Enhancing (also referred to herein as “increasing” or “upregulating”) any of the targets disclosed herein (e.g. tlp, myo-inositol catabolism pathway) can be effected at the genomic level (i.e., activation of transcription via promoters, enhancers, regulatory elements), at the transcript level (i.e., activation of translation) or at the protein level (i.e., post-translational modifications, interaction with substrates and the like).

According to specific embodiments, the agent is a polynucleotide.

Enhancing expression of a polypeptide by genome editing, transformation or transfection can be achieved by: (i) replacing an endogenous sequence encoding the polypeptide of interest or a regulatory sequence under the control which it is placed, and/or (ii) inserting a new gene encoding the polypeptide of interest in a targeted region of the genome, and/or (iii) introducing point mutations which result in up-regulation of the gene encoding the polypeptide of interest (e.g., by altering the regulatory sequences such as promoter, enhancers, 5′-UTR and/or 3′-UTR, or mutations in the coding sequence). Thus, according to specific embodiments, the agent capable of enhancing expression of a target disclosed herein is an exogenous polynucleotide sequence designed and constructed to express at least a functional portion of the target.

To express exogenous polynucleotide in a cell, a polynucleotide sequence encoding the target) is preferably ligated into a nucleic acid construct suitable for cell expression. Such a nucleic acid construct includes a promoter sequence for directing transcription of the polynucleotide sequence in the cell in a constitutive or inducible manner.

Constitutive promoters suitable for use with some embodiments of the invention are promoter sequences, which are active under most environmental conditions and most types of cells such as the cytomegalovirus (CMV) and Rous sarcoma virus (RSV). Inducible promoters suitable for use with some embodiments of the invention include for example the tetracycline-inducible promoter (Zabala M, et al., Cancer Res. 2004, 64(8): 2799-804) or pathogen-inducible promoters. Such promoters include those from pathogenesis-related proteins (PR proteins), which are induced following infection by a pathogen.

According to specific embodiments, the promoter is a bacterial nucleic acid (e.g., expression) construct.

A bacterial promoter is any DNA sequence capable of binding bacterial RNA polymerase and initiating the downstream (3′) transcription of a coding sequence into mRNA. A promoter can have a transcription initiation region, which is usually placed proximal to the 5′ end of the coding sequence. This transcription initiation region typically includes an RNA polymerase binding site and a transcription initiation site. A bacterial promoter can also have a second domain called an operator, which can overlap an adjacent RNA polymerase binding site at which RNA synthesis begins. The operator permits negative regulated (inducible) transcription, as a gene repressor protein can bind the operator and thereby inhibit transcription of a specific gene. Constitutive expression can occur in the absence of negative regulatory elements, such as the operator. In addition, positive regulation can be achieved by a gene activator protein binding sequence, which, if present is usually proximal (5′) to the RNA polymerase binding sequence.

An example of a gene activator protein is the catabolite activator protein (CAP), which helps initiate transcription of the lac operon in Escherichia coli (Raibaud et al. (1984) Annu. Rev. Genet. 18:173). Regulated expression can therefore be either positive or negative, thereby either enhancing or reducing transcription. Other examples of positive and negative regulatory elements are well known in the art. Various promoters that can be included in the protein expression system include, but are not limited to, a T7/LacO hybrid promoter, a trp promoter, a T7 promoter, a lac promoter, and a bacteriophage lambda promoter. Any suitable promoter can be used to carry out the present invention, including the native promoter or a heterologous promoter. Heterologous promoters can be constitutively active or inducible. A non-limiting example of a heterologous promoter is given in U.S. Pat. No. 6,242,194 to Kullen and Klaenhammer.

Sequences encoding metabolic pathway enzymes provide particularly useful promoter sequences. Examples include promoter sequences derived from sugar metabolizing enzymes, such as galactose, lactose (lac) (Chang et al. (1987) Nature 198:1056), and maltose. Additional examples include promoter sequences derived from biosynthetic enzymes such as tryptophan (trp) (Goeddel et al. (1980) Nucleic Acids Res. 8:4057; Yelverton et al. (1981) Nucleic Acids Res. 9:731; U.S. Pat. No. 4,738,921; EPO Publication Nos. 36,776 and 121,775). The beta-lactamase (bla) promoter system (Weissmann, (1981) “The Cloning of Interferon and Other Mistakes,” in Interferon 3 (ed. I. Gresser); bacteriophage lambda PL (Shimatake et al. (1981) Nature 292:128); the arabinose-inducible araB promoter (U.S. Pat. No. 5,028,530); and T5 (U.S. Pat. No. 4,689,406) promoter systems also provide useful promoter sequences. See also Balbas (2001) Mol. Biotech. 19:251-267, where E. coli expression systems are discussed.

In addition, synthetic promoters that do not occur in nature also function as bacterial promoters. For example, transcription activation sequences of one bacterial or phage promoter can be joined with the operon sequences of another bacterial or phage promoter, creating a synthetic hybrid promoter (U.S. Pat. No. 4,551,433). For example, the tac (Amann et al. (1983) Gene 25:167; de Boer et al. (1983) Proc. Natl. Acad. Sci. 80:21) and trc (Brosius et al. (1985) J. Biol. Chem. 260:3539-3541) promoters are hybrid trp-lac promoters comprised of both trp promoter and lac operon sequences that are regulated by the lac repressor. The tac promoter has the additional feature of being an inducible regulatory sequence. Thus, for example, expression of a coding sequence operably linked to the tac promoter can be induced in a cell culture by adding isopropyl-1-thio-.beta.-D-galactoside (IPTG). Furthermore, a bacterial promoter can include naturally occurring promoters of non-bacterial origin that have the ability to bind bacterial RNA polymerase and initiate transcription. A naturally occurring promoter of non-bacterial origin can also be coupled with a compatible RNA polymerase to produce high levels of expression of some genes in prokaryotes. The phage T7 RNA polymerase/promoter system is an example of a coupled promoter system (Studier et al. (1986) J. Mol. Biol. 189:113; Tabor et al. (1985) Proc. Natl. Acad. Sci. 82:1074). In addition, a hybrid promoter can also be comprised of a phage promoter and an E. coli operator region (EPO Publication No. 267,851).

The nucleic acid construct nucleic acid construct (also referred to herein as an “expression vector” or a “vector”) can additionally contain a nucleotide sequence encoding the repressor (or inducer) for that promoter. For example, an inducible vector of the present invention can regulate transcription from the Lac operator (LacO) by expressing the nucleotide sequence encoding the Lad repressor protein. Other examples include the use of the lexA gene to regulate expression of pRecA, and the use of trpO to regulate ptrp. Alleles of such genes that increase the extent of repression (e.g., laclq) or that modify the manner of induction (e.g., lambda CI857, rendering lambda pL thermo-inducible, or lambda CI+, rendering lambda pL chemo-inducible) can be employed.

In the construction of the expression vector, the promoter is preferably positioned approximately the same distance from the heterologous transcription start site as it is from the transcription start site in its natural setting. As is known in the art, however, some variation in this distance can be accommodated without loss of promoter function.

Other than containing the necessary elements for the transcription and translation of the inserted coding sequence, the expression construct of some embodiments of the invention can also include sequences engineered to enhance stability, production, purification, yield or toxicity of the expressed polypeptide.

The nucleic acid construct of some embodiments of the invention includes additional sequences which render this vector suitable for replication and integration in prokaryotes, eukaryotes, or preferably both (e.g., shuttle vectors). In addition, typical vectors may also contain a transcription and translation initiation sequence, transcription and translation terminator and a polyadenylation signal. By way of example, such constructs will typically include a 5′ LTR, a tRNA binding site, a packaging signal, an origin of second-strand DNA synthesis, and a 3′ LTR or a portion thereof.

The expression vector of some embodiments of the invention can further include additional polynucleotide sequences that allow, for example, the translation of several proteins from a single mRNA such as an internal ribosome entry site (IRES) and sequences for genomic integration of the promoter-chimeric polypeptide.

Selectable marker genes that ensure maintenance of the vector in the cell can also be included in the expression vector. Preferred selectable markers include those, which confer resistance to drugs such as ampicillin, chloramphenicol, erythromycin, kanamycin (neomycin), and tetracycline (Davies et al. (1978) Annu. Rev. Microbiol. 32:469). Selectable markers can also allow a cell to grow on minimal medium or in the presence of toxic metabolite and can include biosynthetic genes, such as those in the histidine, tryptophan, and leucine biosynthetic pathways.

Where appropriate, the polynucleotides may be optimized for increased expression in the transformed organism. For example, the polynucleotides can be synthesized using preferred codons for improved expression.

Various methods known within the art can be used to introduce the expression vector of some embodiments of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, natural or induced transformation, lipofection, electroporation and infection with recombinant viral vectors. In addition, see U.S. Pat. Nos. 5,464,764 and 5,487,992 for positive-negative selection methods.

Exemplary methods of introducing expression vectors into bacterial cells include for example conventional transformation or transfection techniques, or by phage-mediated infection. As used herein, the terms “transformation”, “transduction”, “conjugation”, and “protoplast fusion” are intended to refer to a variety of art-recognized techniques for introducing foreign nucleic acid (e.g., DNA) into a cell, such as calcium chloride co-precipitation.

Introduction of nucleic acids by phage infection offers several advantages over other methods such as transformation, since higher transfection efficiency can be obtained due to the infectious nature of phages.

An agent capable of enhancing a target disclosed herein may also be any compound which is capable of increasing the transcription and/or translation of an endogenous DNA or mRNA encoding the target and thus increasing endogenous activity.

A non-limiting example of such an agent is an agent, which inhibits expression or activity of a repressor of the target.

Thus, for example, an agent capable of enhancing myo-inositol catabolism is a compound, which inhibits expression of activity of iolR, as further disclosed hereinabove and in the Examples section hereinbelow.

An agent capable of upregulating a target disclosed herein (e.g. tlp) may also be an exogenous polypeptide including at least a functional portion (as described hereinabove) of the target.

According to specific embodiments, the enhancing agent is a small molecule.

According to specific embodiments, the enhancing agent is an antibody.

Upregulation of a target can be also achieved by introducing at least one substrate. Non-limiting examples of such agents include myo-inositol, inositol or a catabolic product thereof for enhancing myo-inositol catabolism, as further disclosed hereinabove.

According to specific embodiments, the agent of some embodiments of the present invention reduce or prevent formation of a biofilm and/or disrupt a biofilm.

As used herein, the term “biofilm” refers to an aggregate of living microorganisms which are stuck to each other and/or immobilized onto a surface as colonies. The present inventors have uncovered that the biofilm comprises a structured calcium carbonate lamina. The microorganisms in a biofilm are typically embedded within a self-secreted matrix of extracellular polymeric substance (EPS), which is a polymeric sticky mixture of nucleic acids, proteins and polysaccharides. The cells of a microorganism growing in a biofilm are physiologically distinct from cells in the “planktonic form” of the same organism, which by contrast, are single-cells that may float or swim in a liquid medium. Biofilms can go through several life-cycle steps which include initial attachment, irreversible attachment, one or more maturation stages, and dispersion.

Biofilms may comprise a single microbial species or may be mixed species complexes.

Methods of detecting and analyzing a biofilm are well known in the art and include, but are not limited to, microscopy (e.g. Atomic Force Microscopy, Transmitting Electron Microscopy, Scanning Transmitting Electron Microscopy, light microscopy, epifluorescence microscopy, scanning electron microscopy, confocal microscopy), histology, histochemistry, immunohistochemistry, micro-CT, X-ray diffraction (XRD) and FTIR.

Biofilms may form on a wide variety of biological and non-biological surfaces including, but not limited to, living tissues, medical devices, hospital and lab equipment, industrial or potable water system piping, or natural aquatic systems.

Hence, contacting a biofilm-producing microorganism with the agent can be performed in-vivo, in-vitro or ex-vivo.

According to other specific embodiments, contacting is effected in-vivo.

According to specific embodiments, contacting is effected in-vitro or ex-vivo

As used herein, the term “reducing formation of a biofilm” refers to a decrease in the appearance of a biofilm by a biofilm-producing microorganism as compared to same in the absence of the agent, as may be manifested by e.g. reduced mass, reduced rate of buildup of a biofilm, increased permeability or increased sensitivity to an anti-microbial agent, reduced loss of function of infected tissues and devices; and may be determined by e.g. micro-CT, FTIR, microscopy histochemistry and/or immunohistochemistry.

According to specific embodiment, reducing formation of a biofilm assumes that the biofilm has not yet been formed.

Alternatively or additionally, specific embodiments of the present invention disclose that a biofilm has already been formed and the agent reduces the biofilm growth.

According to specific embodiments, the decrease in formation of a biofilm is at least 1.5 fold, at least 2 fold, at least 3 fold, at least 5 fold, at least 10 fold, or at least 20 fold as compared to same in the absence of the agent.

According to other specific embodiments the decrease in formation of a biofilm is by at least 5%, by at least a 10%, at least 20%, at least 30%, at least 40%, at least 50%, at least 60%, at least 70%, at least 80%, at least 90%, at least 95% or at least 99% as compared to same in the absence of the agent.

As used herein, the term “preventing formation of a biofilm” refers to keeping the appearance of a biofilm from occurring in a situation wherein a biofilm has not yet been established or matured, as may be manifested by e.g. reduced mass, increased permeability or increased sensitivity to an anti-microbial agent, reduced loss of function of infected tissues and devices; and may be determined by e.g. micro-CT, FTIR, microscopy histochemistry and/or immunohistochemistry.

As used herein, the term “disrupting” refers to a decrease in an established or matured biofilm as compared to prior to contacting the biofilm-producing microorganism with the agent, and may be determined by e.g. micro-CT, FTIR, microscopy histochemistry and/or immunohistochemistry.

According to specific embodiments the decrease is at least 1.5 fold, at least 2 fold, at least 3 fold, at least 5 fold, at least 10 fold, or at least 20 fold as compared to prior to contacting with the agent.

According to other specific embodiments the decrease is by at least 5%, by at least a 10%, at least 20%, at least 30%, at least 40%, at least 50%, at least 60%, at least 70%, at least 80%, at least 90%, at least 95% or at least 99% as compared to prior to contacting with the agent.

According to specific embodiments, disrupting a biofilm results in converting at least a portion of the biofilm (e.g., at least 5%, 10%, 20%, 30%, 40%, 50%, 60%, 70%, 80%, 90% and even 100%) into planktonic cells.

As specific applications of the invention assumes reducing or preventing formation of a biofilm wherein a biofilm as not yet been formed, while other application of the invention assumes reducing formation of a biofilm or disrupting a biofilm wherein a biofilm has been established, the agent of some embodiments of the present invention may be introduced prior to, during or following the detection of a biofilm.

Hence, according to specific embodiments, contacting is effected prior to formation of a biofilm.

According to specific embodiments, contacting is effected following formation of a biofilm.

Specific embodiments of the invention disclose that the agent (e.g. carbonic anhydrase inhibitor and/or the urease inhibitor) does not affect planktonic cells.

Thus, according to specific embodiments, the agent (e.g. carbonic anhydrase inhibitor and/or the urease inhibitor) affects the biofilm but is not cytotoxic to the microorganism.

According to specific embodiments, contacting with the agents (e.g. carbonic anhydrase inhibitor and/or the urease inhibitor) is effected at a non-cytotoxic dose to the microorganism.

As used herein, the term “microorganism” or “biofilm-producing microorganism” refers to any microorganism capable of producing a biofilm and include, but is not limited to, bacterium, fungi, algae, protozoa, archaea and the like.

According to specific embodiments, the microorganism is pathogenic.

According to other specific embodiments, the microorganism is not pathogenic.

According to specific embodiments, the microorganism is a bacterium.

Hence, according to specific embodiments, the biofilm is a bacterial biofilm.

According to specific embodiments, the bacterium is a gram positive bacterium.

According to specific embodiments, the bacterium is a gram negative bacterium.

According to specific embodiments, the bacterium is Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia and/or Xanthomonas, each possibility represents a separate embodiment of the present invention.

According to specific embodiments, the bacterium is Pseudomonas aeruginosa, Enterococcus faecalis, Staphylococcus aureus, Proteus mirabolis, Pathogenic Escherichia coli or Salmonella Typhimurium, each possibility represents a separate embodiment of the present invention.

According to specific embodiments, the bacterium is Pseudomonas aeruginosa.

According to specific embodiments, the bacterium is not Helicobacter Pylori.

According to specific embodiments, the biofilm does not comprise Helicobacter Pylori.

According to specific embodiments, the bacterium is selected from the group consisting of Bacillus simplex, Bacillus simplexmegaterium, Bacillus sp., Bacillus brevis and Bacillus licheniformis.

According to specific embodiments, the microorganism is a fungi.

According to specific embodiments, the fungi is Candida, Aspergillus, Cryptococcus, Trichosporon, Coccidioides, and/or Pneumocystis, each possibility represents a separate embodiment of the present invention.

According to specific embodiments, the microorganism is resistant to an anti-microbial drug. The drug-resistant microorganism can be resistant to one or more anti-microbial agents.

As shown in the Examples section which follows (Example 2), chemical inhibition of urease and/or carbonic anhydrase increased biofilm permeability and sensitized the bacteria to bactericides treatment.

Hence, according to an aspect of the present invention, there is provided a method of increasing sensitivity of a biofilm-producing bacteria to an anti-microbial agent, the method comprising contacting the biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when said agent is said carbonic anhydrase inhibitor or said urease inhibitor said at least 1 agent is at least 2 agents, thereby increasing sensitivity of the biofilm-producing bacteria to the anti-microbial agent.

According to an alternative or an additional aspect of the present invention, there is provided a method of increasing sensitivity of a biofilm-producing bacteria to an anti-microbial agent, the method comprising contacting the biofilm-producing microorganism with a carbonic anhydrase inhibitor and a urease inhibitor, thereby increasing sensitivity of the biofilm-producing bacteria to the anti-microbial agent.

According to an alternative or an additional aspect of the present invention, there is provided a method of increasing sensitivity of a biofilm-producing bacteria to an anti-microbial agent, the method comprising contacting the biofilm-producing microorganism with a carbonic anhydrase inhibitor and/or a urease inhibitor, wherein said carbonic anhydrase inhibitor and/or said urease inhibitor is administered at a non-cytotoxic dose to said microorganism, thereby increasing sensitivity of the biofilm-producing bacteria to the anti-microbial agent.

According to an alternative or an additional aspect of the present invention, there is provided a method of increasing sensitivity of a biofilm-producing bacteria to an anti-microbial agent, the method comprising contacting the biofilm-producing microorganism with at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby increasing sensitivity of the biofilm-producing bacteria to the anti-microbial agent.

According to an alternative or an additional aspect of the present invention, there is provided a method of increasing sensitivity of a biofilm-producing bacteria to an anti-microbial agent, the method comprising contacting the biofilm-producing microorganism with a carbonic anhydrase inhibitor and/or a urease inhibitor and an antimicrobial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby increasing sensitivity of the biofilm-producing bacteria to the anti-microbial agent.

As used herein the term “increasing sensitivity” refers to an increase of at least 5% in a microorganism's susceptibility to an anti-microbial agent, as compared to same in the absence of the agent, as may be manifested e.g. in growth arrest and/or death. According to a specific embodiment, the increase is in at least 10%, 20%, 30%, 40% or even higher say, 50%, 60%, 70%, 80%, 90% or more than 100%.

According to embodiments of the present invention, any of the agents presented herein may be used in combination with an additional active agent, or alternatively, in a composition including an additional active agent.

When using an additional active agent in any of the methods and uses described herein, the additional agent can be contacted with the microorganism or otherwise administered to the subject, concomitantly, concurrently, simultaneously, consecutively or sequentially with the agent (e.g. carbonic anhydrase inhibitor, urease inhibitor, Ca²⁺ ATPase) described herein.

According to specific embodiments, the additional active agent is an anti-microbial agent.

Thus, according to specific embodiments, the methods of the present invention comprise contacting the microorganism with an anti-microbial agent.

As used herein, the phrase “anti-microbial agent” refers to an agent which affects the growth of a microbial population. The anti-microbial agent can be cytotoxic or cytostatic to the microorganism and/or the microbial population.

According to specific embodiments, the anti-microbial agent is a cytotoxic agent.

According to specific embodiments, the anti-microbial agent is a disinfectant or an antiseptic.

Non-limiting examples of disinfectants and antiseptics which are suitable for use in the context of some embodiments of the present invention include chlorine, active oxygen, iodine, alcohols, phenolic substances, cationic surfactants, strong oxidizers, heavy metals, strong acids and alkalis.

According to specific embodiments, the anti-microbial agent is an anti-bacterial agent (e.g. a bactericide e.g. an antibiotic).

Non-limiting examples of anti-bacterial agents which are suitable for use in the context of some embodiments of the present invention include, amikacin, amoxicillin, ampicillin, azithromycin, aztreonam, cefazolin, ceftriaxone, cefepime, cefonicid, cefotetan, ceftazidime, cephalosporin, cephamycin, chloramphenicol, chlortetracycline, ciprofloxacin, clarithromycin, clindamycin, colistin, cycloserine, dalfopristin, doxycycline, ephalothin, erythromycin, gatifloxacin, gentamicin, imipenem, kanamycin, levofloxacin, lincosamide, linezolid, meropenem, moxifloxacin, mupirocin, neomycin, nitrofurantoin, oxacillin, oxytetracycline, piperacillin, penicillin, quinupristin, rifampicin, spectinomycin, streptomycin, sulfanilamide, sulfamethoxazole, tazobactam, tetracycline, tobramycin, trimethoprim and vancomycin, as well as any of combinations and any derivatives thereof.

According to specific embodiments, the anti-microbial agent is selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to specific embodiments, the anti-microbial agent is an anti-fungal agent.

Non-limiting examples of anti-fungal agents which are suitable for use in the context of some embodiments of the present invention include, amphotericin, amphotericin B, nystatin and pimaricin, amphotericin B liposomal formulations (AmBisome, Abelcet, Amphocil), azole-based antifungal agents such as fluconazole, itraconazole and ketoconazole, allylamine- or morpholine-based antifungal agents such as allylamines (naftifine, terbinafine), and antimetabolite-based antifungal agents such as 5-fluorocytosine, and fungal cell wall inhibitor such as echinocandins like caspofungin, micafungin and anidulafungin, as well as any of combinations and any derivatives thereof.

According to specific embodiments, the anti-microbial agent is not amphotericin B.

According to specific embodiments, the anti-microbial agent is not a beta-lactone.

According to specific embodiments, the anti-microbial agent is not a beta-lactam.

According to specific embodiments, the anti-microbial agent is not an anti-microbial peptide, such as disclosed e.g. in U.S. Pat. No. 9,243,036.

As mentioned hereinabove and shown in the Examples section which follows (Examples 2 and 5), chemical inhibition of urease, carbonic anhydrase and/or Ca²⁺ ATPase inhibited assembly of complex bacterial structures, prevented formation of protective diffusion barriers, increased biofilm permeability and sensitized the bacteria to bactericides treatment. Further, deletion of the Ca²⁺ ATPase YloB or iolR prevented biofilm formation whereas deletion of Tlp increased calcification and biofilm formation (Example 4).

Consequently, the present teachings further suggest the agents disclosed herein can be used for, but not limited to, treating a medical condition in which reducing or preventing formation of a biofilm and/or disrupting a biofilm is beneficial.

Thus, according to an aspect of the present invention there is provided a method of treating a medical condition in which reducing or preventing formation of a biofilm and/or disrupting a biofilm is beneficial in a subject in need thereof, the method comprising administering to the subject a therapeutically effective amount of at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when said agent is said carbonic anhydrase inhibitor or said urease inhibitor said at least 1 agent is at least 2 agents,

thereby treating the medical condition in the subject.

According to an alternative or an additional aspect of the present invention there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and a urease inhibitor, thereby treating the medical condition in the subject.

According to an alternative or an additional aspect of the present invention there is provided at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

for use in the treatment of a medical condition in which reducing or preventing formation of a biofilm and/or disrupting a biofilm is beneficial,

wherein when said agent is said carbonic anhydrase inhibitor or said urease inhibitor said at least 1 agent is at least 2 agents.

According to an alternative or an additional aspect of the present invention, there is provided a carbonic anhydrase inhibitor and a urease inhibitor for use in the treatment of a medical condition in which reducing or preventing formation of a biofilm and/or disrupting a biofilm is beneficial.

According to an alternative or an additional aspect of the present invention, there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and/or a urease inhibitor, wherein said carbonic anhydrase inhibitor and/or said urease inhibitor is administered at a non-cytotoxic dose to a microorganism producing said biofilm, thereby treating the medical condition in the subject.

According to an alternative or an additional aspect of the present invention, there is provided a carbonic anhydrase inhibitor and/or a urease inhibitor for use in the treatment of a medical condition in which reducing or preventing formation of a biofilm and/or disrupting a biofilm is beneficial, wherein said carbonic anhydrase inhibitor and/or said urease inhibitor is administered at a non-cytotoxic dose to a microorganism producing said biofilm.

According to an alternative or an additional aspect of the present invention, there is provided a method of treating a medical condition in which reducing or preventing formation of a biofilm and/or disrupting a biofilm is beneficial in a subject in need thereof, the method comprising administering to the subject a therapeutically effective amount of at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby treating the medical condition in the subject.

According to an alternative or an additional aspect of the present invention, there is provided a method of treating a medical condition in which reducing or preventing formation of a biofilm and/or disrupting a biofilm is beneficial in a subject in need thereof, the method comprising administering to the subject a therapeutically effective amount of a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby treating the medical condition in the subject.

According to an alternative or an additional aspect of the present invention, there is provided at least 1 agent selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, for use in in the treatment of a medical condition in which reducing or preventing formation of a biofilm and/or disrupting a biofilm is beneficial According to an alternative or an additional aspect of the present invention, there is provided a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, for use in in the treatment of a medical condition in which reducing or preventing formation of a biofilm and/or disrupting a biofilm is beneficial.

As used herein, the phrase “medical condition in which reducing or preventing formation of a biofilm and/or disrupting a biofilm is beneficial” refers to a medical condition wherein at least one adverse manifestation of the medical condition is caused or augmented by a biofilm-producing microorganism and encompasses medical conditions of which the microorganism is the primary cause of the medical condition or a secondary effect of the main medical conditions. Hence, such medical conditions comprise an infection with a biofilm-producing microorganism and/or a biofilm infection.

As used herein, the term “subject” includes mammals, e.g., human beings at any age and of any gender who suffer from the pathology (e.g., a medical condition in which reducing or preventing formation of a biofilm and/or disrupting a biofilm is beneficial). According to specific embodiments, this term encompasses individuals who are at risk to develop the pathology.

The term “treating” or “treatment” refers to inhibiting, preventing or arresting the development of a pathology (e.g., a medical condition in which reducing or preventing formation of a biofilm and/or disrupting a biofilm is beneficial) and/or causing the reduction, remission, or regression of a pathology or a symptom of a pathology. Those of skill in the art will understand that various methodologies and assays can be used to assess the development of a pathology, and similarly, various methodologies and assays may be used to assess the reduction, remission or regression of a pathology. Thus, for example, microbial and/or biofilm infection may be assessed by, but not limited to, clinical evaluation, urine dipstick tests, throat culture, sputum tests, histology, indirect non-culture-based tests, including C-reactive protein and procalcitonin tests, direct non-culture-based tests detect antigens or specific antibodies, serological tests, radiography and/or nucleic acid amplification tests.

Non-limiting examples of such medical conditions include dermatitis, acne, chronic bronchitis, bronchiectasis, aspergillosis, asthma, cystic fibrosis, pneumonia, urinary tract infection, chronic gingivitis, chronic rhinosinusitis; chronic periodontitis, chronic inflammatory bowel disease, chronic eczema, atopic dermatitis, chronic non-healing wounds, chronic cystitis, chronic blepharitis, dry eye syndrome, meibomianitis and rosacea, ear infection, “swimmer's ear”, otitis externa, chronic otitis, chronic sinusitis, chronic tonsillitis, adenoiditis, infectious kidney stones, endocarditis, vaginal infection, gastrointestinal tract infection, prostatitis, dental caries, Legionnaire's disease, osteomyelitis, allergic rhinitis, allergic conjunctivitis, wounds, burns, surgical procedures and device related infection.

According to specific embodiments, the medical condition is selected from the group consisting of chronic otitis media, chronic sinusitis, chronic tonsillitis, dental plaque, chronic laryngitis, endocarditis, lung infection, kidney stones, biliary tract infections, vaginosis, osteomyelitis and chronic wounds.

According to specific embodiments, the medical condition is cystic fibrosis.

According to specific embodiments, the medical condition is a device related infection.

Such devices include, but are not limited to, medical implants, wound care devices, drug delivery devices and body cavity and personal protection devices such as urinary catheters, intravascular catheters, vascular central catheters, peripheral vascular catheters, cannulae, ventricular derivations, dialysis shunts, wound drain tubes, skin sutures, vascular grafts, implantable meshes, intraocular devices, cardiac valves, cardiac stents, pacemakers, biliary stents, wound dressings, contact lenses, biologic graft materials, tissue fillers, breast implants, orthopedic implants, prosthetic joints, cochlear and middle ear implants, endotracheal tubes, tape closures and dressings, surgical incise drapes, needles, drug delivery skin patches, drug delivery mucosal patches, medical sponges, tampons, sponges, surgical and examination gloves, toothbrushes, intrauterine devices (IUDs), diaphragms, condoms and the like.

According to specific embodiments, the agent (e.g. carbonic anhydrase inhibitor, the urease inhibitor and/or the Ca²⁺ ATPase inhibitor) can be administered to a subject in combination with other established or experimental therapeutic regimen to treat a disease (e.g. a medical condition in which reducing or preventing formation of a biofilm and/or disrupting a biofilm is beneficial) including anti-microbial agents, anti-inflammatory agents, cytotoxic therapies, analgesics, hormonal therapy and other treatment regimens which are well known in the art.

Hence, according to specific embodiments, the methods of the present invention comprise administering to the subject an additional therapy for the medical condition, or alternatively, the agents disclosed herein are used in combination with an additional therapy for the medical condition.

According to specific embodiments, the additional therapy for the medical condition comprises an anti-microbial agent.

According to specific embodiments, the agents disclosed herein are not used in combination with beta-lactones.

According to specific embodiments, the agents disclosed herein are not used in combination with beta-lactams.

According to specific embodiments, the agents disclosed herein are not used in combination with an anti-microbial peptide, such as disclosed e.g. in U.S. Pat. No. 9,243,036.

According to specific embodiments, the agents disclosed herein are not used in combination with a pH adjusting agent.

According to specific embodiments, the agents disclosed herein are not used in combination with a pH adjusting agent that maintains a pH of between approximately 1.5 and approximately 6.0 in the microorganism microenvironment.

Each of the agents, anti-microbial agents and therapies for treating the medical conditions described hereinabove can be administered to the individual per se or as part of a pharmaceutical composition which also includes a physiologically acceptable carrier. The purpose of a pharmaceutical composition is to facilitate administration of the active ingredient to an organism.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the agent (e.g. carbonic anhydrase inhibitor, urease inhibitor Ca²⁺ ATPase inhibitor), anti-microbial agent and/or therapy for treating the medical condition accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

The route of administration is selected to suite the medical condition which is being treated. For example, in treating a systemic infection where rapid distribution of is needed, the active agent(s) is typically administered orally or intravenously. When treating a local infection, the active agent(s) is typically administered locally, topically, transdermally, subcutaneously or intramuscularly.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

According to specific embodiments, an exemplary method of treating a medical condition in which reducing or preventing formation of a biofilm and/or disrupting a biofilm is beneficial is effected by applying a solid support coated or attached with the active compound (e.g. implanting a medical device, applying a wound care device topically onto a wound, providing a subcutaneous medical device).

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions, which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by oral and/or nasal inhalation, the active ingredients for use according to some embodiments of the invention are conveniently delivered in the form of an aerosol spray presentation from a pressurized pack, metered dose inhaler or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch. For administration by oral inhalation the active ingredients for use according to some embodiments of the invention can also be delivered by a dry powder inhaler.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents, which increase the solubility of the active ingredients to allow for the preparation of highly concentrated solutions. Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients effective to prevent, alleviate or ameliorate symptoms of a disorder (e.g., a medical condition in which reducing or preventing formation of a biofilm and/or disrupting a biofilm is beneficial) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

According to specific embodiments, the agent if administered at a non-cytotoxic dose to the microorganism.

According to specific embodiments, the carbonic anhydrase inhibitor and/or said urease inhibitor are administered at a non-cytotoxic dose to the microorganism.

According to specific embodiments, the antimicrobial agent is administered at a dose below the common gold standard dose.

Dosage amount and interval may be adjusted individually to provide levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

The components necessary to carry out any of the methods described herein may be provided individually or may be comprised in a kit.

Thus, according to an aspect of the present invention there is provided an article of manufacture comprising at least 2 agents selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator.

According to an alternative or an additional aspect of the present invention there is provided an article of manufacture comprising a carbonic anhydrase inhibitor and a urease inhibitor.

According to specific embodiments, the article of manufacture further comprises an anti-microbial agent.

According to an alternative or an additional aspect of the present invention there is provided an article of manufacture comprising at least 1 agent selected from the group consisting of:

(i) a carbonic anhydrase inhibitor;
(ii) a urease inhibitor;
(iii) a Ca²⁺ ATPase inhibitor;
(iv) a tlp activator; and
(v) a myo-inositol catabolism pathway activator,

and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an alternative or an additional aspect of the present invention there is provided an article of manufacture comprising a carbonic anhydrase inhibitor and/or a urease inhibitor and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole. According to specific embodiments, the agents comprised in the article of manufacture (e.g. the carbonic anhydrase inhibitor, the urease inhibitor and/or the anti-microbial agent) are packaged in separate containers.

According to yet other specific embodiments, at least 2 of the agents comprised in the article of manufacture (e.g. the carbonic anhydrase inhibitor, the urease inhibitor and/or the anti-microbial agent) are in a co-formulation.

As mentioned, biofilms may form on a wide variety of biological and non-biological surfaces. Consequently, the carbonic anhydrase inhibitor and/or urease inhibitor in the methods and/or the articles of manufacture of some embodiments of the present invention are coating or attached to a solid support, either alone or in combination with an additional active agent (e.g., an anti-microbial agent).

Thus, according to an aspect of the present invention there is provided an article of manufacture comprising:

(a) a solid support; and

(b) at least 1 agent coating or attached to said solid support, wherein said at least 1 agent is selected from the group consisting of:

(i) a carbonic anhydrase inhibitor;

(ii) a urease inhibitor;

(iii) a Ca²⁺ ATPase inhibitor;

(iv) a tlp activator; and

(v) a myo-inositol catabolism pathway activator,

wherein when said agent is said carbonic anhydrase inhibitor or said urease inhibitor said at least 1 agent is at least 2 agents.

According to an alternative or an additional aspect of the present invention there is provided an article of manufacture comprising:
(i) a solid support; and
(ii) a carbonic anhydrase inhibitor and a urease inhibitor coating or attached to said solid support.

According to specific embodiments, the article of manufacture further comprises an anti-microbial agent coating or attached to the solid support.

According to an alternative or an additional aspect of the present invention there is provided an article of manufacture comprising:

(a) a solid support;

(b) at least 1 agent coating or attached to said solid support, wherein said at least 1 agent is selected from the group consisting of:
(i) a carbonic anhydrase inhibitor;

(ii) a urease inhibitor;
(iii) a Ca²⁺ ATPase inhibitor;
(iv) a tlp activator; and
(v) a myo-inositol catabolism pathway activator; and

(c) an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

According to an alternative or an additional aspect of the present invention there is provided an article of manufacture comprising:

(i) a solid support;

(ii) a carbonic anhydrase inhibitor and/or a urease inhibitor coating or attached to said solid support; and

(iii) an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole.

Solid support that can be used with specific embodiments of the present invention include any surface, structure, product or material, which can support, harbor or promote the growth of a biofilm. Such products include, for example, medical devices, hospital and lab equipment, food products, agricultural products, cosmetic products, industrial or potable water system piping or natural aquatic systems and the like. Non-limiting examples include an implantable medical device (such as a gastric or duodenal sleeve, urinary catheter, intravascular catheter, dialysis shunt, wound drain tube, skin suture, vascular graft, implantable mesh, intraocular device, heart valve, stents, orthopedic implants and the like), a topical medical device such as a wound care device (such as general wound dressings, biologic graft materials, tape closures and dressings, surgical incise drapes, contact lenses and the like), a subcutaneous medical device (such as a subcutaneous injection port, a percutaneous medical device such as a catheter, a syringe needle), a drug delivery device (such as a needle, drug delivery skin patch, drug delivery mucosal patch, medical sponge and the like), a body cavity and personal protection device (such as tampon, sponge, surgical and examination glove, toothbrush, intrauterine devices (IUDs), diaphragms, condom and the like), an endoscopic device, a vessel, a tube, a lid, a wrap, a package, a work surface or area, a warehouse, a package and the like, the inner walls of a storage container that is routinely treated with anti-microbial agents, a soil and/or soil enrichment supplements, an agricultural product or crop, a cosmetic product, a building, warehouse, compartment, container or transport vehicle, a dye or a paint and any other materials and industrial compounds which require protection of their surfaces against microorganisms, such as, for example, construction materials.

Coating or attaching the active agents described herein (e.g. carbonic anhydrase inhibitor, urease inhibitor, anti-microbial agent) to the solid support is effected typically by dipping, spraying, impregnating, flushing or otherwise applying the active agent(s) or a composition comprising the same, as described herein, in or on the solid support. Such methods are known in the art and described e.g. in U.S. Pat. Nos. 4,107,121; 4,442,133; 4,895,566; 4,917,686; 5,013,306; 5,624,704; 5,688,516; 5,756,145; 5,853,745; 5,902,283; 6,719,991.

As shown in the Examples section which follows, the present inventors used a high-resolution and robust μCT technique to study the mineralized areas within intact bacterial biofilms and discovered that the structure of the calcium carbonate deposits can be used to predict biofilm permeability and hence sensitivity to anti-microbial agents (Examples 1-2).

Hence, according to an aspect of the present invention there is provided a method of predicting sensitivity of a biofilm to an anti-microbial agent, the method comprising determining a concentration and/or thickness of a layer of calcium carbonate within the biofilm, wherein a concentration of said calcium carbonate and/or a thickness of a layer of said calcium carbonate above a predetermined threshold indicates said biofilm is resistant to the anti-microbial agent. As used herein the phrase “predetermined threshold” refers to a concentration of calcium carbonate in a biofilm and/or a thickness of a layer of calcium carbonate in a biofilm that characterizes a microbial biofilm sensitive an anti-microbial agent. Such a level can be experimentally determined by comparing resistant biofilms with sensitive biofilms of the same origin. Alternatively, such a level can be obtained from the scientific literature and from databases.

According to specific embodiments, the predetermined threshold is 0.5%, 0.25%, 0.1%, 0.05%, 0.025% or 0.1% calcium carbonate.

According to specific embodiments, the predetermined threshold is 0.5% calcium carbonate.

According to specific embodiments, the predetermined threshold is 0.05% calcium carbonate.

According to specific embodiments, the predetermined threshold is 1 μm, 0.5 μm, 0.1 μm or 0.05 μm of a calcium carbonate layer.

According to specific embodiments, the predetermined threshold is 0.5 μm of a calcium carbonate layer.

According to specific embodiments, determining is effected in-vivo in a subject diagnosed with a biofilm infection.

According to specific embodiments, determining is effected in-vitro or ex-vivo on a biofilm sample obtained from a subject diagnosed with a biofilm infection.

Non-limiting examples of such biofilm samples include a biopsy sample, a surgery samples and a sputum sample.

Determining may be effected by methods well known in the art including, but not limited to, micro-CT, FTIR, TGA analysis, immunostaining.

Typically, a Micro-CT can analyze samples of 100 μM in diameter and an FTIR can analyze less than 0.1 mg of bleached material.

According to specific embodiments, determining is effected by micro-CT.

According to specific embodiments, the determining is effected by a 2D analysis.

According to specific embodiments, the determining is effected by a 3D analysis.

As mentioned hereinabove and shown in the Examples section which follows (Examples 2, 4 and 5), inhibition of urease, carbonic anhydrase, Ca²⁺ ATPase or iolR inhibited calcification and biofilm formation while inhibition of Tlp increased calcification and biofilm formation (Example 4). Although frequently associated with disease, biofilms are also important for engineering applications, such as bioremediation, biocatalysis and microbial fuel cells. Thus, the present invention also contemplates agents for increasing formation of biofilm and/or biomineralization.

Hence, according to an aspect of the present invention, there is provided a method of inducing or increasing formation of a biofilm and/or biomineralization, the method comprising contacting a biofilm-producing microorganism with at least 1 agent selected from the group consisting of:

(i) a Ca²⁺ ATPase activator;

(ii) a tlp inhibitor; and

(iii) a myo-inositol catabolism pathway inhibitor,

thereby inducing or increasing formation of the biofilm and/or the biomineralization.

According to specific embodiments, the method comprising contacting said microorganism with an agent selected from the group consisting of a carbonic anhydrase activator and a urease activator.

As used herein, the phrase “inducing or increasing formation of a biofilm and/or biomineralization” refers to an increase in the appearance of a biofilm or an amount of biomineralization by a biofilm-producing microorganism as compared to same in the absence of the agentr, as may be manifested by e.g., increased mass, increased rate of buildup of a biofilm, decreased permeability or increased amount of calcite; and may be determined by e.g. micro-CT, FTIR, microscopy histochemistry and/or immunohistochemistry.

According to specific embodiment, inducing formation of a biofilm assumes that the biofilm has not yet been formed.

Alternatively or additionally, specific embodiments of the present invention disclose that a biofilm has already been formed and the agent increases the biofilm growth.

According to specific embodiments the increase in formation of a biofilm and/or biomineralization is at least 1.5 fold, at least 2 fold, at least 3 fold, at least 5 fold, at least 10 fold, or at least 20 fold as compared to same in the absence of the agent.

According to other specific embodiments the increase in formation of a biofilm and/or biomineralization is by at least 5%, by at least a 10%, at least 20%, at least 30%, at least 40%, at least 50%, at least 60%, at least 70%, at least 80%, at least 90%, at least 95% or at least 99% as compared to same in the absence of the agent.

Hence, according to specific embodiments, contacting is effected prior to formation of a biofilm.

According to specific embodiments, contacting is effected following formation of a biofilm.

As used herein, the phrase “Ca²⁺ ATPase activator” refers to an agent capable of increasing Ca²⁺ ATPase expression and/or catalytic activity.

According to specific embodiments, the Ca²⁺ ATPase activator increases Ca²⁺ ATPase expression.

According to specific embodiments, the Ca²⁺ ATPase activator increases Ca²⁺ ATPase activity.

As used herein, the phrase “tlp inhibitor” refers to an agent capable of an agent capable of binding tlp or a polynucleotide encoding same and inhibiting its expression or activity.

According to specific embodiments, the tlp inhibitor inhibits tlp expression.

According to specific embodiments, the tlp inhibitor inhibits tlp activity.

As used herein, the phrase “myo-inositol catabolism pathway inhibitor” refers to an agent capable of inhibiting myo-inositol catabolism by affecting expression, activity and/or an amount of any of any of the components involved in myo-inositol catabolism.

According to specific embodiments, the myo-inositol catabolism pathway inhibitor inhibits expression of an enzyme involved in myo-inositol catabolism.

According to specific embodiments, the myo-inositol catabolism pathway inhibitor inhibits expression and/or activity of the iol regulon.

According to specific embodiments, the myo-inositol catabolism pathway inhibitor increases expression and/or activity of iolR.

As used herein, the phrase “carbonic anhydrase activator” refers to an agent capable of increasing carbonic anhydrase expression and/or catalytic activity.

According to specific embodiments, the carbonic anhydrase activator increase carbonic anhydrase expression.

According to specific embodiments, the carbonic anhydrase activator increase carbonic anhydrase activity.

As used herein, the phrase “urease activator” refers to an agent capable of increasing urease expression and/or catalytic activity.

According to specific embodiments, the urease activator increases urease expression.

According to specific embodiments, the urease activator increases urease activity. Specific embodiments of the present invention comprise a single agent selected from a Ca²⁺ ATPase activator; a tlp inhibitor; and a myo-inositol catabolism pathway inhibitor.

Other specific embodiments of the present invention comprise at least 2 or 3 agents selected from (i) a Ca²⁺ ATPase activator; (ii) a tlp inhibitor; and (iii) a myo-inositol catabolism pathway inhibitor.

Thus, specific embodiments of this aspect of the present invention comprise (i)+(ii), (i)+(iii), (ii)+(iii), (i)+(ii)+(iii), each possibility represents a separate embodiment of the present invention.

A detailed description of inhibitory and enhancing agents, which can be used according to specific embodiments of the invention, is provided hereinabove.

The present invention also contemplates microorganisms comprising the agents described herein.

Thus, according to an aspect of the present invention, there is provided a microorganism obtainable by the method.

Such methods and microorganisms can be used for any application wherein formation of a biofilm and/or biomineralization is desired. Such applications are known to the skilled in the art, and disclosed for examples in Wood et al. Trends Biotechnol. 2011 February; 29(2): 87-94; and Qureshi et al. Microb Cell Fact. 2005; 4: 24, the contents of which are fully incorporated herein by reference.

Thus, according to an aspect of the present invention there is provided an industrial product selected from the group consisting of a water cleaning system, a bioremediation system, a microbial leaching system, a biofilm reactor, a microbial fuel cell (MFC), a construction material and a biologic glue, comprising the microorganism obtainable by the method.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first, indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods for Examples 1-2 and 5

Strains and Media—B. subtilis NCIB 3610 (Branda et al. [Branda et al., Proceedings of the National Academy of Sciences of the United States of America (2001) 98, 11621-11626], M. smegmatis MC2155 and P. aeruginosa were grown at 30° C. in B4 medium (0.4% yeast extract, 05% glucose) [Barabesi et al. Journal of bacteriology (2007) 189, 228-235] supplemented with calcium acetate as indicated. Acetohydroxamic acid (AHA) was purchased from Merck (Cat. Num. 159034). FilmTracer™ Calcein Green Biofilm Stain (Cat. Num. F10322) was purchased from ThermoFisher Scientific. DTNB was purchased from Sigma Aldrich (Cat No. D8130). Sodium vanadate, an inhibitor of Ca2+-ATPase [Clausen, J et al. Structure 24, 617-623 (2016)], was obtained from Sigma Aldrich (Cat No. 590088 or 72060).

Growth Analysis—To determine growth kinetics of planktonic bacteria,the cultures were grown for 15-30 hours at 30° C. in a plate reader (BioTek, Winooski, Vt., USA) and the optical density at 600 nm (OD600) was measured every 20 minutes. Results are presented as averages for 5-6 wells from a single representative experiment out of three.

Micro-CT X-ray analysis—To create a 3D reconstruction, a bacterial colony was grown on biofilm-inducing agar medium for the indicated time. The whole, unfixed colony was transferred to a plastic slide, and rotated between the X-ray source and the detector positioned at optimal distances for a cubic voxel size of 0.87 μm. The drying of the sample under X-ray was prevented by mounting the plate in a sealed cell under saturated water vapors atmosphere. 2D projections were taken at different angles over 180°. Images at the indicated magnification were taken using a Zeiss micro XCT 400 instrument (Pleasanton, Calif., USA). Tomography was carried out using a micro-focused source set at 20 kV and 100 μA. 1200 separate 2D images were taken with a pixel size of 0.87 mm over 1800, exposure time of 30 sec. The volume was reconstructed from the complete set of images. Raw data were reconstructed with Zeiss software (Zeiss) that uses a filtered back-projection algorithm. 3D volume rendering (maximum intensity projection) was carried out with Avizo software (VSG, Hillsboro, Oreg., USA).

Imaging—All images were taken using a Nikon D3 camera or a Stereo Discovery V20″ microscope (Tochigi, Japan) with objectives Plan Apo S ×0.5 FWD 134 mm or Apo S ×1.0 FWD 60 mm (Zeiss, Goettingen, Germany) attached to a high-resolution microscopy Axiocam camera. Data were created and processed using Axiovision suite software (Zeiss). To visualize colony cross-sections, a warm low-melt agar was poured over the colony following incubation with FITC, and allowed to solidify at room temperature (RT). Following, slices were made with a razor blade and immediately visualized. All experiments were repeated at least 3 times, in technical triplicates, with similar results.

Flow Cytometry—To determine the intracellular calcium levels, 3 day-old colonies were collected, sonicated gently to remove extracellular matrix and incubated for 1 hour with FilmTracer™ Calcein Green Biofilm Stain prepared and diluted according to manufacturers' instructions. Following, cells were washed 3 times in PBS and sorted by a flow cytometer. Data was acquired with SORP-LSRII flow cytometer (BD Biosciences) and analyzed with BD FACSDIVA™ software. The experiment was repeated 3 times, in technical duplicates, with similar results.

Analysis of the weight of the minerals—(A) Thermogravimetric analysis (TGA) of the B. subtilis colonies was performed as described previously [Levi-Kalisman et al., J Chem Soc Dalton (2000), 3977-3982] for at-least three colonies materials combined for each time point. The weight loss associated with the calcite relates to the temperature range 650-800° C. Results are presented as an average of at-least three independent experiments. (B) Weight of the mineral in a pellicle was determined as described by Mahamid et al. [Proceedings of the National Academy of Sciences of the United States of America (2008) 105, 12748-12753] et al., with some modifications: pellicle samples were slightly bleached with 3% sodium hypochlorite for 1 minute to remove organic matter, washed twice with Milli-Q water (Merck KGaA, Darmstadt, Germany) and dehydrated in acetone. The experiment was repeated 3 times, in technical triplicates, with similar results.

Fourier transform infrared (FTIR) spectrophotometer analysis—FTIR spectra of the produced crystals were acquired in KBr pellets using a NICOLET iS5 spectrometer. A few milligrams of each sample were homogenized and powdered in an agate mortar and pestle. About 0.3 mg were left in the mortar and mixed with about 40 mg of KBr and pressed into a 7 mm pellet using a manual hydraulic press (Specac). Each sample was measured repeatedly by repetitive grinding of the same KBr pellet. Typically, few seconds of regrinding were applied. Infrared spectra were obtained at 4 cm¹ resolution for 32 scans using a Nicolet 380 instrument (Thermo). The baselines for the heights measurements of the v2, v3, and v4 peaks were determined as done previously [Politi Y et al. Science (2004) 306: 1161-1164]. The v2, v3, and v4 heights were normalized to a, v3 height of 1000, corresponding to 1.0 absorbance unit.

Crystal Violet—From each individual culture, 20 μl samples from exponential phase and 180 μl of TSB broth were dispensed in the wells of sterile 96-wells flat-bottomed microtiter plate (Nunc) and incubated at 37° C. for 24 hours. The control well contained only TSB broth without inoculation. Following incubation, unbound cells were removed by inversion of the microtiter plate, followed by vigorous tapping on an absorbent paper. Subsequently, adhered cells were fixed for 30 minutes at 800° C. Adhered cells were stained by addition of 220 μl of crystal violet (0.5%) for 5 minutes. The stain was removed by exhaustive washing with distilled water. The plates were then allowed to dry. In order to quantify adhered cells, 220 μl of decoloring solution (95% ethanol) was added to each well for 15 minutes. The absorption of the eluted stain was measured at 590 nm.

### Example 1

### Extracellular Calcium Carbonate Sheets Serve as Diffusion Barriers in Bacterial Biofilm

Micro-CT which allows obtaining complete 3D information on opaque samples was used to study the detailed inner structure of calcium minerals within biofilm colonies. As this technique detects structured calcium (such as aggregates and crystals), but not amorphous calcium or calcium salts with organic substances, it can be used for identifying and analyzing calcium deposits.

To this end, the development of calcium-rich structures in B. subtilis colonies grown on biomineralization-promoting medium containing 0.25% calcium acetate was evaluated. 3D reconstruction revealed intricate macro-scale structures (FIG. 1A, left panel). The observed structures were highly reminiscent of the biofilm wrinkles in time of development, form and location. Of note, no mineral was produced by B. subtilis colonies grown on media lacking calcium ions (data not shown). Fourier transform infrared (FTIR) spectroscopy was used to identify the precipitated mineral. In this method, IR radiation is passed through a sample when some of it is absorbed by the sample and some of it is transmitted. The resulting spectrum, constitute a unique fingerprint of the sample, represents the molecular absorption and transmission. The organic matter was removed in a hypochlorite priming step, and analysis was performed on the inorganic matter only. The FTIR spectra of the putative calcium carbonate minerals collected from the edges of the biofilm was typical of calcite, a crystalline polymorph of CaCO₃, and differed from other CaCO₃ polymorphs vaterite and aragonite (FIG. 1E).

In order to determine whether these observations reflect general architectural principles, the actinobacterium Mycobacterium smegmatis, which is known to form very robust biofilms²² was also examined. As expected, when the calcium concentration in the growth medium was 0.25% as with B. subtilis, the colony accumulated mineral crystals in a calcium dependent manner making a high-resolution micro-CT X-ray imaging impossible. High-resolution images were thus obtained by lowering the calcium concentration in the medium by ten-fold. Even at low calcium concentration, M. smegmatis formed robust and complex calcium-rich structures (FIG. 1A, left panel), consistent with dense and complicated wrinkles.

The high resolution images enabled segmenting the reconstructed volume, in order to identify and measure calcium in different regions of the colony (FIG. 1A, middle panel). The calcium layer spread over time, and while at day 1 it was mostly observed at the wrinkles, at day 6 most of the colony was covered. Furthermore, the total volume of the colony and of the calcium layer were estimated, and the fraction of the calcium out of the whole colony increased over time (FIG. 1B). This was confirmed by a TGA analysis, which showed accumulation of calcium in the biofilm colonies over time (FIG. 1C).

In the next step virtual transversal slices were generated to investigate the calcium distribution within the sample (FIG. 1A, right panel). The most dense calcium carbonate areas formed a crust-like cover over the wrinkles. Using the parallel plate model the thickness of this layer was calculated—approximately 1 μm for B. subtilis and 3 μm for M. smegmatis. Interestingly, it was found that the thickness of this calcium layer was constant throughout the colony and did not increase during B. subtilis colony development (FIG. 1D). Instead, during biofilm growth the calcium layer spread and covered more and more of the colony wrinkles (see also FIG. 1A, middle panel).

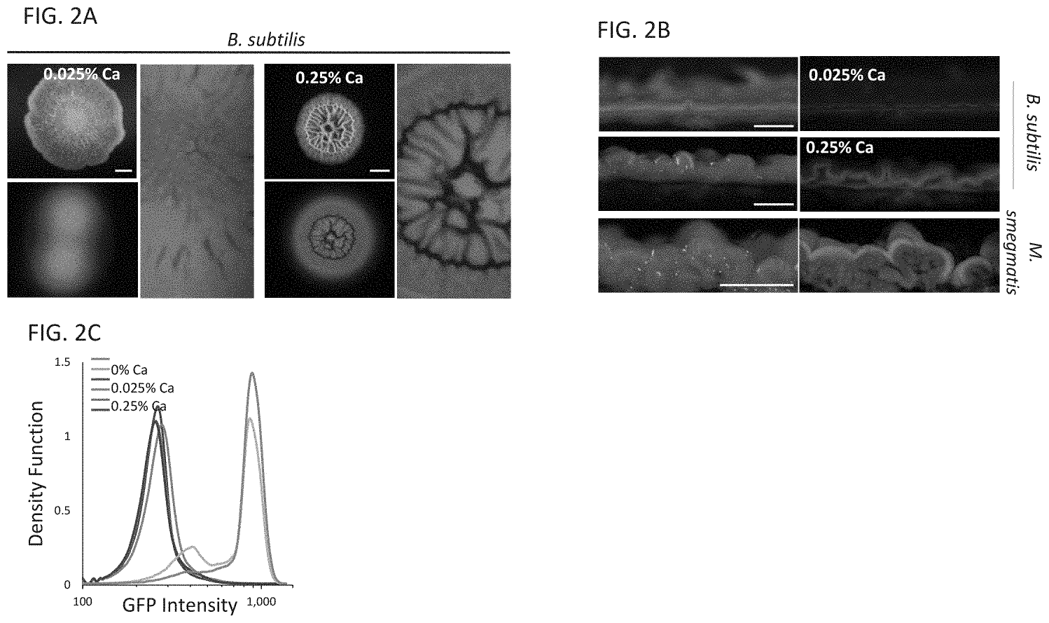

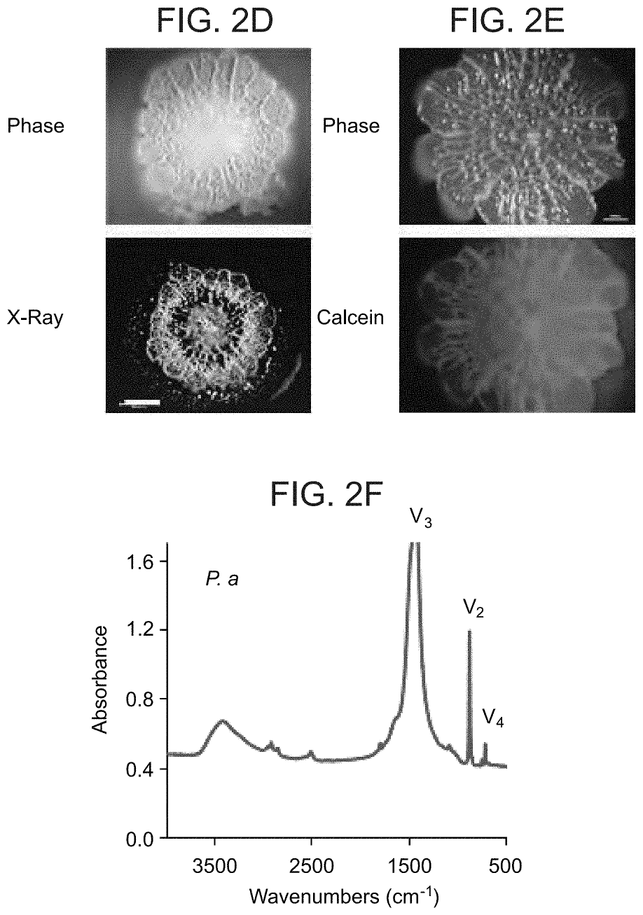

Bacteria within biofilm communities are up to three orders of magnitudes more resistant to antimicrobial agents and the immune system than planktonic bacteria. Hence, while the rigid mineral layer is a structural element, potentially increasing the weight bearing of the wrinkles and supporting the overall colony structure, it might have additional functions. Diffusion of water and small molecular weight solutes is several orders of magnitudes less efficient in calcite^{18,19} compared with organic polymers^{20,23}. Therefore, the present inventors hypothesized that calcium carbonate dense areas could function as diffusion barriers. To this end, the diffusion of water-soluble FITC throughout B. subtilis biofilm colonies, grown in the presence of high (0.25%) and low (0.025%) levels of calcium was evaluated. As shown in FIG. 2A, the diffusion of FITC was limited in the wrinkled biofilm colonies grown in the presence of high calcium concentration; while on the other hand, the dye diffused freely in non-wrinkled colonies formed on low calcium concentration. In order to gain better understanding of the causes of this limitation, the colony was manually sliced and the cross-sections were visualized. As shown in FIG. 2B, in colonies grown in the presence of calcium, the diffusion was limited, and the dye accumulated in a discreet area within the colony biomass. This barrier was calcium dependent, and at low calcium concentrations diffusion was less restricted (FIG. 2B). The clear diffusion barriers and dye accumulation were also evident in M. smegmatis, as would be expected due to its high efficiency in forming biominerals (FIG. 2B). Interestingly, when the intracellular calcium levels were determined by staining cells with calcein AM fluorescent dye, significantly lower levels of intracellular calcium were detected in cells grown at higher calcium concentration (FIG. 2C). This lack of correlation between calcium-dependent morphology and intracellular calcium levels suggests that calcium precipitation occurs in a near proximity, but outside the cells—and is consistent with the current view of calcite biomineralization initiation on the outside of the bacterial membrane²⁴.

The above described biomineralization during biofilm development and the formation of clear diffusion barriers were also evident in a third model organism—Pseudomonas aeruginosa.

As with the other two model organisms, P. aeruginosa colony biofilms grown in the presence of a calcium source were thicker and developed a complex morphology (FIGS. 2D-E). The FTIR spectra of the putative calcium carbonate minerals collected from the edges of the biofilm was typical of calcite (FIG. 2F). In addition, the presence of calcite was confirmed by an independent analysis using X-ray diffraction, and by Calcein staining of the extracellular fraction [using a Calcein non-permeable through the cell membrane].

Taken together, biomineralization is essential for biofilm formation. Formation of extracellular calcium carbonate sheets occurs in parallel to complex colony formation and serves as a diffusion barrier in the bacterial biofilm. Moreover, this phenomenon is conserved and wide-spread in the bacterial kingdom.

### Example 2

### Urease and Carbonic Anhydrase Inhibitors Prevent Biomineralization and the Formation of Protective Diffusion Barriers

The effects of inhibiting activity of two intracellular enzymes which promote calcium carbonate precipitation: carbonic anhydrase and urease on biofilm formation and antibiotic resistance were evaluated.

Carbonic anhydrase is of essential role in biomineralization as CO₂ is converted to carbonic acid by carbonic anhydrase: CO₂+H₂O->H₂CO₃, followed by bicarbonate production H₂CO₃->HCO₃⁻+H⁺.

Additionally, biomineralization associated with microbial metabolism is usually accompanied by increase in environmental alkalinity, which promotes calcium carbonate precipitation¹⁴. One of the central reaction leading to the increased pH is catalyzed by the enzyme urease (FIG. 3A)²⁵. The microbial ureases hydrolyze urea to produce carbonate and ammonia, simultaneously increasing the pH and the carbonate concentration, which then combines with environmental calcium to precipitate as calcium carbonate²⁵.

In the first step, the effect of inhibiting urease activity in B. subtilis and M. smegmatis colonies was evaluated. As shown in FIG. 3B, inhibition of urease activity in B. subtilis by the urease inhibitor acetohydroxamic acid (AHA) lead to smooth colony morphology, even in the presence of calcium, at concentrations having little or no effect on planktonic growth (FIG. 3C, upper panel). In agreement with the hypothesis that biomineralization mechanisms are shared by different taxa, inhibition of urease activity in M. smegmatis inhibited growth, biofilm development and biomineralization. M. smegmatis was more sensitive to urease inhibition, suggesting a central role for urease in the physiology of this bacterium, which would be consistent with its higher rates of biomineralization. However, even AHA concentrations which were well-tolerated by M. smegmatis planktonic cells (FIG. 3C, lower panel), led to less wrinkled colonies, similar to the effect achieved by lowering calcium concentration in the growth medium (FIG. 3D).

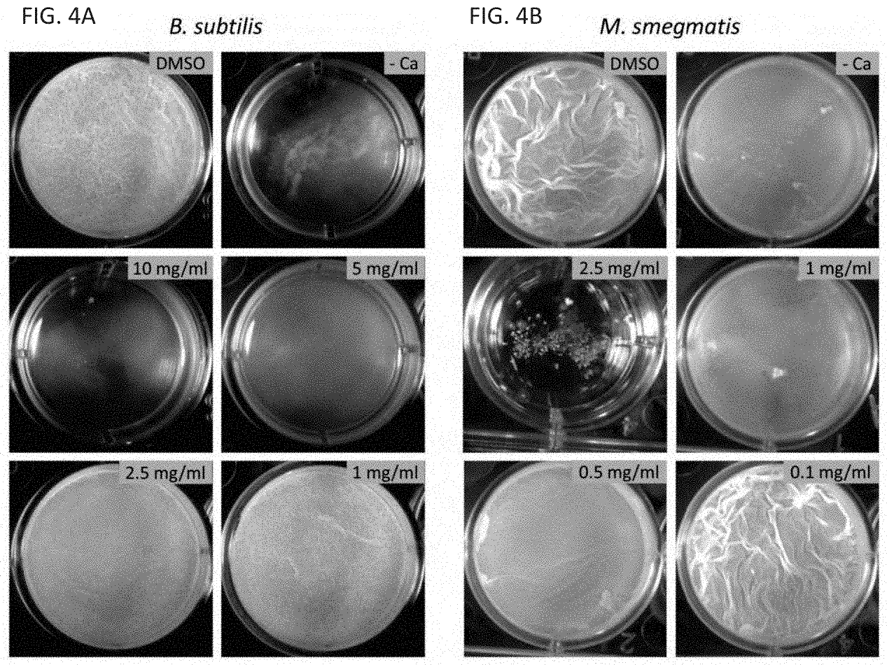

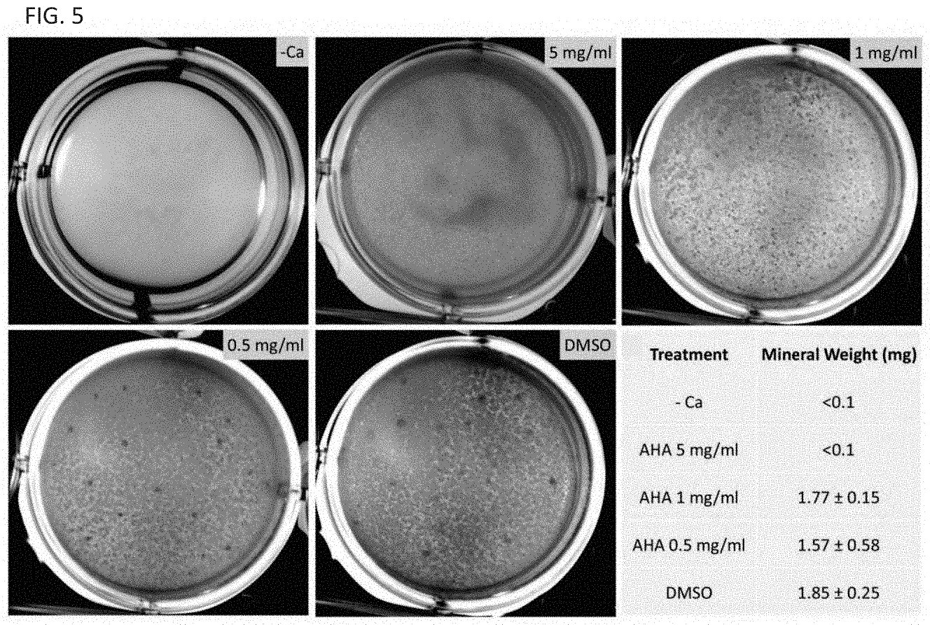

A similar effect on morphology was observed using a pellicle biofilm model system (FIGS. 4A-B). Moreover, inhibition of urease with AHA decreased non-soluble mineral production (FIG. 5). Further, cross-sections of colonies revealed that inhibition of urease with AHA prevented the formation of the diffusion barriers within the colony (FIG. 3E).

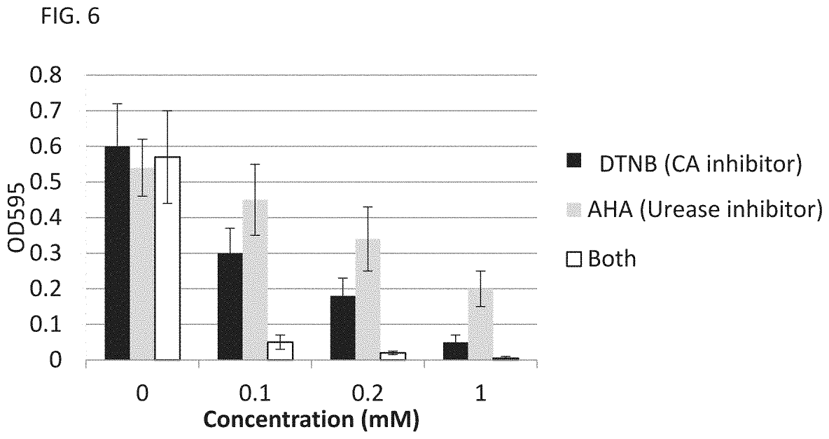

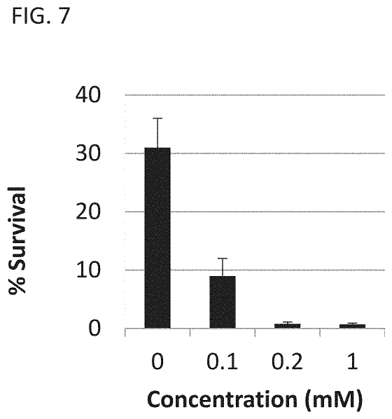

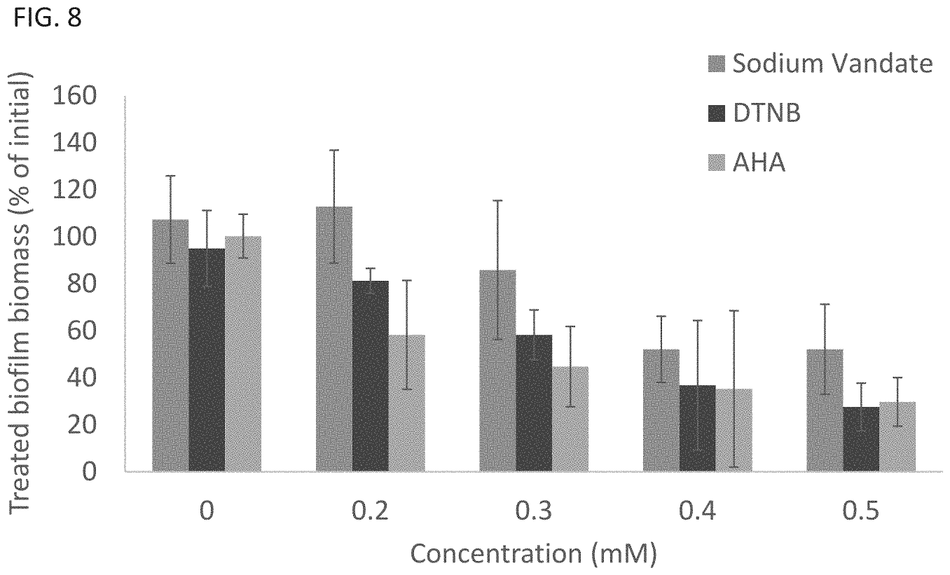

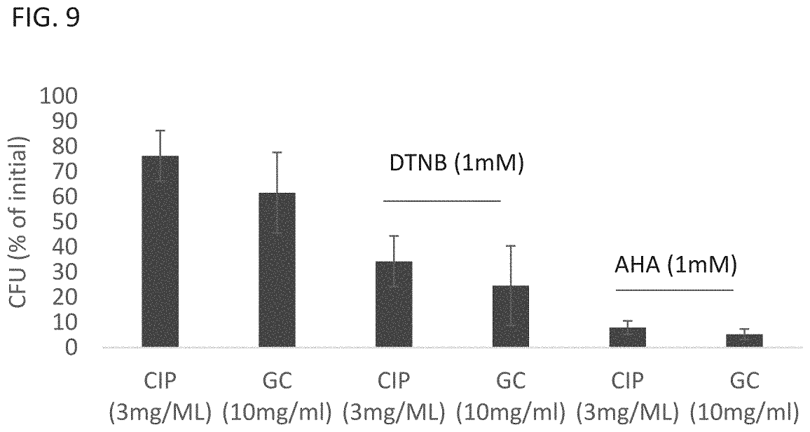

In the second step, the effect of inhibiting urease activity and carbonic anhydrase activity in P. aeruginosa colonies was evaluated. As shown in FIG. 6, inhibition of urease activity by the urease inhibitor acetohydroxamic acid (AHA) or inhibition of carbonic anhydrase activity by DTNB impaired biofilm development. Furthermore, as shown in FIG. 8, treatment with AHA or with DTNB was able to disperse a pre-existing P. aeruginosa biofilm. Moreover, combined treatment with AHA and DTNB had a synergistic effect. Of note, the inhibitors concentrations had little or no effect on planktonic growth (data not shown). Most importantly, treatment with AHA and DTNB, alone or in combination, also sensitized the P. aeruginosa colonies to treatment with ciprofloxacin and gentamicin (FIGS. 7 and 9).

Taken together, chemical inhibition of urease and/or carbonic anhydrase at non-bactericidal doses prevents biomineralization and the formation of protective diffusion barriers, disperse pre-existing biofilm and sensitizes the bacteria to bactericides treatment.

### Example 3

### Urease and Carbonic Anhydrase Inhibitors can be Used for Preventing and/or Treating Colonization of P. aeruginosa Biofilms in Lungs of CF Patients

The following complementary setting are used to evaluate biomineralization in P. aeruginosa biofilms and the effect of urease inhibitors (e.g. AHA, N-(n-butyl)thiophosphoric triamid, ecabet sodium, Epiberberin) and/or carbonic anhydrase inhibitors [e.g. Diamox (Acetazolamide), 5,5′-Dithiobis (2-nitrobenzoic acid, DTNB), sulfumates, sulfamides, brimonidine, N,N-diethyldithiocarbamate, phenylboronic acid, phenylarsonic acid]:

(i) in vitro colony development model [e.g., Kempes, et al. Proceedings of the National Academy of Sciences of the United States of America (2014) 111: 208-213];

(ii) Submerged biofilm model where the biofilm is formed on the bottom of 24 well plates in rich biofilm media [Kolodkin-Gal et al. Science (2010) 328: 627-629] as well as in ASM [Kirchner et al. Journal of visualized experiments: JoVE, (2012) e3857], mimics the composition of the CF septum but is not viscos;

(iii) Ex vivo murine lung model [Massler et al. The journal of gene medicine (2011): 13, 101-113; Kolodkin-Gal, D. et al. Journal of virology (2008) 82: 999-1010; and Kolodkin-Gal, D. et al. Journal of virology (2013) 87: 13589-13597]. For the lung model a GFP fluorescent Pseudomonas aeruginosa wild-type and matrix mutants [Banin et al. Proceedings of the National Academy of Sciences of the United States of America (2005) 102: 11076-11081] are used. Briefly, the lung tissues are cut into 5 mm sections. The cut sections are washed in PBS and placed in a 24 well plates. This size of the explant allows survival of the tissue from one hand, and sufficient surface area for biofilm formation on the other hand. An ASM media is used for infection: In this media the fluorescent P. aeruginosa strains are suspended and placed on the top of the tissue on a final volume of 500 μL for each well. The tissue sections infected with the bacterial cells are further incubated for different time periods, washed, and grown further to assess biofilm development. The development of the biofilm is assessed by e.g. Confocal Laser Scanning Microscopy (CLSM), MicroCT, FTIR analysis Calcein staining.

(iv) Ex vivo human lung model. Similar approach as in (iii) is used to assess P. aeruginosa calcite calcification and biofilm formation in lungs obtained from CF patients, as well as CF patients undergoing lung transplantation. The results are also aligned with the clinical manifestation of the infection, and the burden of P. aeruginosa cells in the lung, quantified using replicative counts of colony forming units on top of an indicative growth media.

Following, similar settings are used to compare sensitivity of the untreated and treated biofilms to bactericidal agents [e.g. disinfectants (e.g. bleach, ethanol), vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam), trimethoprim-sulfamethoxazole]. For Example, a fluorescent derivative of vancomycin-BPD [Bucher, T. Environmental microbiology reports, (2015) doi:10.1111/1758-2229.12346; and Tiyanont, K. et al. Proceedings of the National Academy of Sciences of the United States of America (2006) 103: 11033-11038] is used to assess diffusion into the biofilm in parallel with viability measurements. Viability is determined by e.g. viable counts of the bacteria before and after the treatment, live/dead staining, evaluating cellular integrity of the biofilm cells.

Alternatively or additionally, the effect of urease inhibitors and/or carbonic anhydrase inhibitors on P. aeruginosa activity and viability in the lungs is determined in-vivo in CF patients. For example, the carbonic anhydrase inhibitor Diamox is administered by inhalation to CF patients.

To this end:

(1) Enzymatic activity of carbonic anhydrase is measured in the sputum of patients with CF. Levels are compared between patients with mucoid and non-mucoid pseudomonas chronic infection. Mucoid pseudomonas is characterized by biofilm formation. Deep sputum samples are obtained from patients with CF that come routinely to the CF clinic (after intensive physiotherapy). Sputum is an emerging matrix for potential outcome measures associated with CF lung disease. The standardized method for processing sputum that was developed by the Cystic Fibrosis Therapeutics Development Network (CFTDN) is used to maximize the quality and quantity of data obtained from studying CF sputum. Carbonic anhydrase and urease activity is measured using well-established enzymatic and colorimetric essays and compared according to the clinical status of the patients [Muller, W. E. G. et al. Febs Open Bio (2013) 3: 357-362, Urease activity essay kit, Sigma].

(2) Nasal surface pH is measured by a Sandhill ZepHr PHNS-P (Sandhill Scientific, Highlands Ranch, Colo.) Mobidium pH probe with an internal reference electrode. Prior to each study, the pH probe is calibrated in buffer solutions of pH 6, 7 and 8 (VWR, West Chester, Pa.). Voltage is recorded with an Oakton pH meter (Cole-Parmer, Vernon Hills, Ill.) and corrected to temperature. The probe is positioned 6 cm (adults), 1.5 cm children) from the most caudal aspect of the columella. The catheter remains in position until the reading is stable for 15 s. All measurements are taken by the same operator.

(3) Exhaled breath condensate pH (EBC) is collected using a device, which consisted of a mouthpiece and a two-way non-rebreathing valve connected by polypropylene tubing to a glass Dreschel flask immersed in crushed ice, acting as a condensing chamber. Subjects breath at a normal frequency and tidal volume for 15 minutes while wearing nose clips, allowing collection of 1.5-2.5 ml of condensate. The pH is measured immediately with a benchtop pH meter (Fisher Scientific Instruments,Loughborough, UK).

Following, intravenous preparation of Diamox 500 mg is inhaled and its effect is assessed:

(1) Patients receive one inhalation and the pH is measured prior to and following the inhalation.

(2) Patients inhale Diamox 3 times every day for one month together with their routine antibiotic therapy; and clinical parameters (pulmonary function BMI), pseudomonas growth (number of bacterial colonies) and levels of sputum carbonic anhydrase are measured.

Materials and Methods

Patient's samples—Sputum was collected from adult patients as published previously [N. L. Schiller, R. L. Millard, Pediatr Res 17, 747-752 (1983)] and stored in 4° C. degrees to allow microscopy. All patients positive for calcite were carrying chronic pseudomonas infections. Table 1 hereinbelow provides specific patient information.

TABLE 1

| | Medical | | |
|---|---|---|---|
| Patient | Center | Gender | CFTR mutations |
| A4 | Hadassa | F | W1282X |
| A62 | Hadassa | M | A455E |
| 463123 | Carmel | F | F508del |
| 463125 | Carmel | F | T360K, Q359K |

Ex vivo lung infection system—Lungs were harvested from 2 mice (one month old) and placed in petri dishes containing DMEM 5% FCS. The tissue was divided into circular pieces 4 mm in diameter with a biopsy punch and transferred to a 24 wells plate (4-5 explants/well) with 450 μl DMEM containing 100 μg/ml carbenicillin (Sigma-Aldrich) and 0, 2.5, or 5 mg/ml AHA or DTNB. To each respective well, either 50 μl DMEM (control) or P. aeruginosa pretreated with 0, 2.5, or 5 mg/ml AHA or with 0, 1, 5 or 10 mM DTNB and diluted within DMEM to attain OD₆₀₀ 0.373 or 0.257, respectively, were added, with three technical repeats for each condition. The plates were incubated at 37° C. for ˜2 days (48-52 hours) and washed twice with PBS, fixed with PFA 4% for 10 minutes, and embedded in either cryosection (OCT) compound or paraffin. Paraffin samples were cut into 7 micron slices and stained with H&E; whereas cryosections were cut into 10 micron slices and placed on superfrost plus slides.

Results

P. aeruginosa dependent calcite formation was studied in the sputum of CF patients. The results indicate that for multiple patients, P. aeruginosa biofilms can form calcite crystals during lung infections, and that those crystals are tightly associated with bacterial cells (FIG. 10A-C). To determine whether biochemical pathways involved in biofilm calcification can be targeted to combat persistent infection, a novel ex vivo system, where lung tissues are harvested, and then infected with P. aeruginosa was developed (FIG. 10D). Clear biofilms were formed on the tissue prior to its consumption (FIG. 10D). The effect of AHA and DTNB was tested in this system. As shown in FIGS. 10D and 11, treatment with AHA or DTNB lead to diminished lung colonization by P. aeruginosa and prevented lung tissue death.

### Example 4

### Deletion of iolR or YloB Prevents Biomineralization and Biofilm Formation and Deletion of Tlp Increases Biomineralization and Biofilm Formation

Materials and Methods

Strains and media—All strains were derivatives from wild type Bacillus subtilis NCIB 3610. Additional laboratory strains such as B. subtilis PY79 and Escherichia coli DH5α were used for cloning purposes. Table 2 hereinbelow shows the list of strains used. Deletions were generated by long-flanking PCR mutagenesis. A list of primers used for cloning is shown in Table 3 hereinbelow. Transformation of B. subtilis PY79 with double-stranded PCR fragments was done as described previously [Z. Bloom-Ackermann et al., Environmental microbiology 18, 5032-5047 (2016)].

B. subtilis biofilms were grown on B4 biofilm-promoting solid medium (0.4% yeast extract, 0.5% glucose, and 1.5% agar) [C. Barabesi et al., Journal of bacteriology 189, 228-235 (2007)] supplemented with calcium acetate as indicated, incubated at 30° C. in a sealed box for enriched CO₂ environment achieved by using the candle jar method [Y. Oppenheimer-Shaanan et al., NPJ biofilms and microbiomes 2, 15031 (2016)].

TABLE 2

| Strain # | Strain | Genotype |
|---|---|---|
| | B. subtilis | Wild type |
| | NCIB 3610 | |
| IKG0893 | B. subtilis | ΔiolR |
| | NCIB 3610 | |
| IKG0895 | B. subtilis | ΔyloB |
| | NCIB 3610 | |
| IKG0897 | B. subtilis | Δtlp |
| | NCIB 3610 | |
| | P. aeruginosa | pMRP9-1 |
| | PA14 | (Ppuc-GFP) |

TABLE 3

| SEQ | | |
|---|---|---|
| ID | | |
| NO | Primer | Sequence |
| 1 | M005 | TCCTGAGCCTGTTGCTTAACCCGGTC |
| | yloB A | |
| 2 | M006 | CAATTCGCCCTATAGTGAGTCGTTCC |
| | yloB B | GCTCGTCCACTCCCCTGCTC |
| 3 | M007 | CCAGCTTTTGTTCCCTTTAGTGAGAT |
| | yloB C | TCATATGATATAATCTTAGGGGTAAT |
| | | AGCG |
| 4 | M008 | CCTAGTAAGACCGTCTGTAAACAATA |
| | yloB D | CGC |
| 5 | M017 | ATGCTAGTCGGTTATACGGGATG |
| | tlp A | |
| 6 | M018 | CAATTCGCCCTATAGTGAGTCGTTCG |
| | tlp B | GATGTACCTCCTAGAAATAAACTCG |
| 7 | M019 | CCAGCTTTTGTTCCCTTTAGTGAGGG |
| | tlp C | ATACCGTTCTTAAAAAACCAGGG |
| 8 | M020 | GATCGGTTAGGTTTTCTTGTTCCA |
| | tlp D | |
| 9 | M038 | CGCTTTAATTTCGGGATGCTCGAGGA |
| | iolR A | |
| 10 | M039 | CAATTCGCCCTATAGTGAGTCGTATA |
| | iolR B | AAAAACTCCTTCTTGAATCTTTACG |
| 11 | M040 | CCAGCTTTTGTTCCCTTTAGTGAGCG |
| | iolR C | TTTACAATAGTGTTGAGAGTCTATCA |
| | | TCC |
| 12 | M041 | CTGATATTTTCTTTGAAACGCTCACC |
| | iolR D | C |

Scanning electron microscopy and EDX—Biofilm colonies were grown for 1, 3, 6, 10 and 15 days at 30° C. on biofilm-promoting B4 solid medium, with or without calcium. The colonies were fixed overnight at 4° C. with 2% glutaraldehyde, 3% paraformaldehyde, 0.1 M sodium cacodylate (pH 7.4) and 5 mM CaCl₂, dehydrated and dried as described by Bucher et al. [Journal of visualized experiments: JoVE, (2016); and Environmental microbiology reports, (2015)]. Clinical samples were first partially or completely bleached to remove the organic material by 1 hour of incubation in either 3% or 6% sodium hypochlorite, respectively. The insoluble material was collected, washed three times in PBS, three times in acetone and air-dried for 16 hours. Mounted samples were coated with 15 nm thick carbon layer in carbon coater (EDVARDS). The imaging by secondary electron (SE) or back scattered electron (BSE) detectors and the Energy Dispersive X-ray Spectroscopy (EDS, Bruker) were preformed using a high-resolution Carl Zeiss Ultra 55 or Supra scanning electron microscopes.

Cryo-STEM analysis—Bacterial colonies grown as described were suspended in PBS buffer. Quantifoil TEM grids were glow-discharged with an Evactron Combi-Clean glow-discharge device, and 5 microliters of suspended cells were deposited onto the glow-discharged grids. Ten nm-diameter gold fiducials [L. Duchesne, et al. Langmuir 24, 13572-13580 (2008)] were applied before blotting and vitrification using a Leica EM-GP automated plunging device (Leica). Vitrified samples were observed with a Tecnai F20 S/TEM instrument at 200 kV, with Gatan 805 brightfield and Fischione HAADF detectors. Microscope conditions: extraction voltage=4300 V, gun lens=3 or 6, and spot size=5 or 6 with 10 micrometer condenser apertures, yielding probe diameters of 1-2 nm and semi-convergence angles of ˜1.3-2.7 mrad. Images of 2048×2048 pixels were recorded with probe dwell times of 8-18 microseconds. Spatial sampling was set between 1 and 4 nm/pixel. Electron doses were 1-3 electrons/A² per dwell spot. Single-axis tilt series were recorded using SerialEM [J. R. Kremer, et al. Journal of structural biology 116, 71-76 (1996)]. EDX was performed in STEM mode on vitrified cell samples with the same electron microscope set-up as used for STEM imaging, using a liquid N2 cooled Si(Li) detector (EDAX).

Tomography reconstructions and visualization—The tomographic tilt series were aligned using fiducial markers and reconstructed using weighted back projection (9) (as implemented in the IMOD software suite (8) (Reconstructions are displayed after non-linear anisotropic diffusion filtering within IMOD. Segmentation and volume rendering were performed using Amira 6.3 (FEI Visualization Sciences Group).

RNA extraction and library preparation—Biofilm colonies were grown on biofilm-promoting B4 solid medium with and without calcium for 1, 2, 3, 6 and 10 days. Three independent experiments were conducted, with three colonies from each treatment combined for RNA extraction in each experiment. The samples were frozen in liquid nitrogen and stored until extraction. Frozen bacterial pellets were lysed using the Fastprep homogenizer (MP Biomedicals) and RNA was extracted with the FastRNA PROT blue kit (MP Biomedicals, 116025050) according to the manufacturer's instructions. RNA levels and integrity were determined by Qubit RNA BR Assay Kit (Life Technologies, Q10210) and TapeStation, respectively. All RNA samples were treated with TURBO DNase (Life Technologies, AM2238).

A total of 5 μg RNA from each sample was subjected to rRNA depletion using the Illumina Ribo-Zero rRNA Removal Kit (Bacteria, MRZB12424), according to the manufacturers' protocols. RNA quantity and quality post-depletion was assessed as described above. RNA-seq libraries were contracted with NEBNext® Ultra™ Directional RNA Library Prep Kit (NEB, E7420) according to the manufacturer's instructions. Libraries concentrations and sizes were evaluated as above, and were sequenced as multiplex indexes in one lane using the Illumina HighSeq2500 platform.

RNAseq processing—Reads were trimmed from their adapter with cutadapt and aligned to the B. subtilis genome (subsp. subtilis str. NCIB 3610, NZ_CM000488.1) with Bowtie2 version 2.3.4.1 [B. Langmead, S. L. Nat Methods 9, 357-359 (2012)]. The number of uniquely mapped reads per gene were calculated using HT-seq [S. Anders, et al. Bioinformatics 31, 166-169 (2015)]. Normalization and testing for differential expression was performed with DESeq2 version 1.16. A gene was considered to be differentially expressed using the following criteria: normalized mean read count ≥30, fold change ≥3, and adjusted p value <0.05. First, differential expression was tested between samples grown with and without calcium separately for each time point; however, since the results for days 1, 2 and 3 were the same days 1-3 were joined.

Comparison between growth conditions—The RNAseq expression data was compared to publically available 269 transcriptomes representing 269 different growth conditions [P. Nicolas et al., Science 335, 1103-1106 (2012)]. Because that study used microarray platform and not RNAseq, from every condition and every replicate the top 10% genes with the highest expression level were extracted (383 genes). Following, the Jaccard index was used to measure the overlap between the conditions of the two platforms (i.e. the current study and (12). Prior to the analysis, 152 genes that appear among the top 10% in more than 80% of the conditions were removed.

Regulon analysis—To analyze the data by regulons, the definition of Subtiwiki [R. H. Michna, et al. Nucleic acids research 44, D654-662 (2016)] was used. For every regulon, the average expression for days 1-3 was calculated using the differentially expressed genes that are associated with the regulon. Out of 215 regulons that are defined in Subtiwiki, 83 were found to contain differentially expressed genes.

Phase microscopy—Biofilm colonies were observed using a Nikon D3 camera or a Stereo Discovery V20″ microscope (Tochigi, Japan) with objectives Plan Apo S×0.5 FWD 134 mm or Apo S×1.0 FWD 60 mm (Zeiss, Goettingen, Germany) attached to a high-resolution microscopy Axiocam camera, as required. Data were captured using Axiovision suite software (Zeiss).

Thermogravimetric (TGA) analysis—Biofilm colonies were grown on biofilm-promoting B4 solid medium at 30° C. for 14 days, with or without calcium. Samples were collected and lyophilized for 24 hours. Dried samples were analyzed by SDT Q 600 (TA Instruments) according to the manufacturer's instructions. The weight loss associated with the calcite relates to the temperature range 650-800 C°. Results are an average of three independent experiments.

Planktonic growth assays—All strains were grown from a single colony isolated over LB plates to a mid-logarithmic phase of growth (4 h at 37° C. with shaking). Cells were diluted 1:100 in 150 μl liquid B4 medium with and without calcium in 96-wells microplate (Thermo Scientific). Cells were grown at 30° C. or 37° C. for 14-20 hours in a microplate reader (Synergy 2, BioTek), and the optical density at 600 nm (OD₆₀₀) was measured every 15 minutes. Three independent experiments were conducted, with three technical repeats per plate.

Results

As shown in Example 1 hereinabove, the formation of extracellular calcium carbonate sheets is calcium dependent. To gain an insight into the metabolic and biochemical pathways involved in the formation of mineral structures and their potential regulation, the effect of calcium on the transcriptome of the biofilm cells was analyzed in B. subtilis. Following, functionally of the several novel pathways and genes identified by the transcriptome analysis was assessed:
The transcriptome analysis showed that the myo-inositol-3-phosphate catabolism regulon (iolR regulon) members were downregulated by calcium (FIG. 12A). Subsequently, deletion of iolR (the transcriptional repressor of the iolR regulon) and artificial upregulation of this regulon yielded B. subtilis colonies defective in their 3D architecture, lacking a response to calcium, and significantly reduced the formation of crystalline calcium carbonate (FIGS. 12C and 12E).
The transcriptome analysis showed that YloB, a calcium ATPase, was upregulated by calcium (FIG. 12B. Subsequently, deletion of this gene dramatically reduced B. subtilis biofilm formation, further strengthening the importance of calcification to biofilm development (FIGS. 12C-E).
The transcriptome analysis showed that expression of Tlp, a highly charged thioredoxin-like protein, was significantly altered (FIG. 12B). Subsequently, deletion of tlp lead to calcium-dependent changes in B. subtilis biofilm morphology, and increased calcification (FIGS. 12C and 12E).

Deletion of iolR, yloB and tlp did not affect planktonic growth either with or without calcium (FIG. 12F), suggesting that they are specifically required for biofilm development.

### Example 5

### Urease, Carbonic Anhydrase and Ca2+-ATPase Inhibitors Inhibit Biofilm Formation

The effect of inhibiting urease activity (using AHA), carbonic anhydrase activity (using DTNB) and Ca2+-ATPase activity (using Sodium vanadate) in P. aeruginosa colonies was evaluated. As shown in FIGS. 13-14, all inhibitors inhibited biofilm formation. In addition, the sodium vanadate was synergistic with DTNB and with AHA.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### REFERENCES

### Other References are Cited Throughout the Application

1 Stoodley, P., Sauer, K., Davies, D. G. & Costerton, J. W. Biofilms as complex differentiated communities. Annual review of microbiology 56, 187-209, doi:10.1146/annurev.micro.56.012302.160705 (2002).
2 Miller, M. B. & Bassler, B. L. Quorum sensing in bacteria. Annual review of microbiology 55, 165-199, doi:10.1146/annurev.micro.55.1.165 (2001).
3 Kolter, R. & Greenberg, E. P. Microbial sciences: the superficial life of microbes. Nature 441, 300-302, doi:10.1038/441300a (2006).
4 Costerton, J. W., Stewart, P. S. & Greenberg, E. P. Bacterial biofilms: a common cause of persistent infections. Science 284, 1318-1322 (1999).
5 Bryers, J. D. Medical biofilms. Biotechnology and bioengineering 100, 1-18, doi:10.1002/bit.21838 (2008).
6 Branda, S. S., Vik, S., Friedman, L. & Kolter, R. Biofilms: the matrix revisited. Trends in microbiology 13, 20-26, doi:10.1016/j.tim.2004.11.006 (2005).
7 Branda, S. S., Gonzalez-Pastor, J. E., Ben-Yehuda, S., Losick, R. & Kolter, R. Fruiting body formation by Bacillus subtilis. Proceedings of the National Academy of Sciences of the United States of America 98, 11621-11626, doi:10.1073/pnas.191384198 (2001).
8 Li, X. et al. Spatial patterns of carbonate biomineralization in biofilms. Applied and environmental microbiology 81, 7403-7410, doi:10.1128/AEM.01585-15 (2015).
9 Li, X. et al. In Situ Biomineralization and Particle Deposition Distinctively Mediate Biofilm Susceptibility to Chlorine. Applied and environmental microbiology 82, 2886-2892, doi:10.1128/AEM.03954-15 (2016).
10 Li, X., Lu, N., Brady, H. R. & Packman, A. I. Biomineralization strongly modulates the formation of Proteus mirabilis and Pseudomonas aeruginosa dual-species biofilms. FEMS microbiology ecology, doi:10.1093/femsec/fiw189 (2016).
11 Li, X., Lu, N., Brady, H. R. & Packman, A. I. Ureolytic Biomineralization Reduces Proteus mirabilis Biofilm Susceptibility to Ciprofloxacin. Antimicrobial agents and chemotherapy 60, 2993-3000, doi:10.1128/AAC 0.00203-16 (2016).
12 Oppenheimer-Shaanan, Y. et al. Spatio-temporal assembly of functional mineral scaffolds within microbial biofilms. NPJ Biofilms Microbiomes 2, 15031, doi:10.1038/npjbiofilms 0.2015.31 (2016).
13 Dade-Robertson, M., Keren-Paz, A., Zhang, M. & Kolodkin-Gal, I. Architects of nature: growing buildings with bacterial biofilms. Microbial biotechnology, doi:10.1111/1751-7915.12833 (2017).
14 Dhami, N. K., Reddy, M. S. & Mukherjee, A. Biomineralization of calcium carbonates and their engineered applications: a review. Frontiers in microbiology 4, 314, doi:10.3389/fmicb.2013.00314 (2013).
15 Perito, B. & Mastromei, G. Molecular basis of bacterial calcium carbonate precipitation. Progress in molecular and subcellular biology 52, 113-139, doi:10.1007/978-3-642-21230-7_5 (2011).
16 Dupraz, C. et al. Processes of carbonate precipitation in modern microbial mats. Earth-Sci Rev 96, 141-162, doi:DOI 10.1016/j.earscirev.2008.10.005 (2009).
17 Ibrahim, M. & Issa, M. Evaluation of chloride and water penetration in concrete with cement containing limestone and IPA. Constr Build Mater 129, 278-288, doi:10.1016/j.conbuildmat.2016.10.085 (2016).
18 Fisler, D. K. & Cygan, R. T. Cation diffusion in calcite: determining closure temperatures and the thermal history for the Allan Hills 84001 meteorite. Meteoritics & planetary science 33, 785-789 (1998).
19 Fisler, D. K. & Cygan, R. T. Diffusion of Ca and Mg in calcite. Am Mineral 84, 1392-1399 (1999).
20 Li, T. Q., Henriksson, U., Klason, T. & Odberg, L. Water Diffusion in Wood Pulp Cellulose Fibers Studied by Means of the Pulsed Gradient Spin-Echo Method. J Colloid Interf Sci 154, 305-315, doi:Doi 10.1016/0021-9797(92)90145-C (1992).
21 Wang, Q. Q., Zhang, C. F., Chu, C. H. & Zhu, X. F. Prevalence of Enterococcus faecalis in saliva and filled root canals of teeth associated with apical periodontitis. International journal of oral science 4, 19-23, doi:10.1038/ijos 0.2012.17 (2012).
22 Purdy, G. E., Pacheco, S., Turk, J. & Hsu, F. F. Characterization of mycobacterial triacylglycerols and monomeromycolyl diacylglycerols from Mycobacterium smegmatis biofilm by electrospray ionization multiple-stage and high-resolution mass spectrometry. Analytical and bioanalytical chemistry 405, 7415-7426, doi:10.1007/s00216-013-7179-4 (2013).
23 Davies, E. et al. Dynamics of water in agar gels studied using low and high resolution 1H NMR spectroscopy. Int J Food Sci Tech 45, 2502-2507, doi:10.1111/j.1365-2621.2010.02448.x (2010).
24 Barabesi, C. et al. Bacillus subtilis gene cluster involved in calcium carbonate biomineralization. Journal of bacteriology 189, 228-235, doi: 10.1128/JB 0.01450-06 (2007).
25 Curthoys, N. P. & Watford, M. Regulation of glutaminase activity and glutamine metabolism. Annual review of nutrition 15, 133-159, doi:10.1146/annurev.nu.15.070195.001025 (1995).

What is claimed is:

1. A method of reducing or preventing formation of a biofilm and/or disrupting a biofilm, the method comprising contacting a biofilm-producing microorganism with at least 1 agent selected from the group consisting of:
(i) a carbonic anhydrase inhibitor;
(ii) a urease inhibitor;
(iii) a Ca²⁺ ATPase inhibitor;
(iv) a tlp activator; and
(v) a myo-inositol catabolism pathway activator,
wherein when said agent is said carbonic anhydrase inhibitor or said urease inhibitor said at least 1 agent is at least 2 agents,
thereby reducing or preventing formation of the biofilm and/or disrupting the biofilm.

2. The method of claim 1, wherein said agent is administered at a non-cytotoxic dose to said microorganism.

3. A method of reducing or preventing formation of a biofilm and/or disrupting a biofilm, the method comprising contacting a biofilm-producing bacteria with a carbonic anhydrase inhibitor and/or a urease inhibitor, wherein said carbonic anhydrase inhibitor and/or said urease inhibitor is administered at a non-cytotoxic dose to said microorganism, thereby reducing or preventing formation of the biofilm and/or disrupting the biofilm.

4. The method of claim 1, comprising contacting said microorganism with an anti-microbial agent.

5. A method of reducing or preventing formation of a biofilm and/or disrupting a biofilm, the method comprising contacting a biofilm-producing bacteria with at least 1 agent selected from the group consisting of:
(i) a carbonic anhydrase inhibitor;
(ii) a urease inhibitor;
(iii) a Ca²⁺ ATPase inhibitor;
(iv) a tlp activator; and
(v) a myo-inositol catabolism pathway activator,
and an anti-microbial agent selected from the group consisting of vancomycin, rifampicin, spectinomycin, cephalosporins (e.g. ceftriaxone-cefotaxime, ceftazidime), fluoroquinolones (e.g. ciprofloxacin, levofloxacin), aminoglycosides (e.g. gentamicin, amikacin), imipenem, broad-spectrum penicillins with or without β-lactamase inhibitors (e.g. amoxicillin-clavulanic acid, piperacillin-tazobactam) and trimethoprim-sulfamethoxazole, thereby reducing or preventing formation of the biofilm and/or disrupting the biofilm.

6. The method of claim 1, being effected in-vitro or ex-vivo.

7. The method of claim 1, begin effected in-vivo.

8. The method of claim 1, wherein said biofilm is a bacterial biofilm.

9. The method of claim 1, wherein said microorganism is a bacterium.

10. The method of claim 9, wherein said bacterium is selected from the group consisting of Acinetobacter, Aeromonas, Bordetella, Brevibacillus, Brucella, Bacteroides, Burkholderia, Borelia, Bacillus, Campylobacter, Capnocytophaga, Cardiobacterium, Citrobacter, Clostridium, Chlamydia, Eikenella, Enterobacter, Escherichia, Entembacter, Francisella, Fusobacterium, Flavobacterium, Haemophilus, Kingella, Klebsiella, Legionella, Listeria, Leptospirae, Moraxella, Morganella, Mycoplasma, Mycobacterium, Neisseria, Pasteurella, Proteus, Prevotella, Plesiomonas, Pseudomonas, Providencia, Rickettsia, Stenotrophomonas, Staphylococcus, Streptococcus, Streptomyces, Salmonella, Serratia, Shigella, Spirillum, Treponema, Veillonella, Vibrio, Yersinia and Xanthomonas.

11. The method or the agent of claim 1, wherein said microorganism is not Helicobacter Pylori.

12. The method of claim 1, wherein said agent and/or said inhibitor is a small molecule.

13. The method of claim 12, wherein said urease inhibitor is selected from the group consisting of AHA, N-(n-butyl)thiophosphoric triamid, ecabet sodium and Epiberberin.

14. The method of claim 12, wherein said carbonic anhydrase inhibitor is selected from the group consisting of Acetazolamide, 5,5′-Dithiobis (2-nitrobenzoic acid, DTNB), sulfumates, sulfamides, brimonidine, N,N-diethyldithiocarbamate, phenylboronic acid and phenylarsonic acid.

15. The method of claim 1, wherein said Ca²⁺ ATPase is YloB.

16. The method of claim 12, wherein said Ca²⁺ ATPase inhibitor is selected from the group consisting of sodium vanadate, EGTA and 1,2-bis(o-aminophenoxy)ethane-N,N,N′,N′-tetraacetic acid (BAPTA), N,N,N′,N′-tetrakis(2-pyridylmethyl)ethane-1,2-diamine (TPEN) and phenanthroline.

17. The method of claim 1, wherein said agent and/or said inhibitor is an antibody, a peptide or an aptamer.

18. The method of claim 1, wherein said myo-inositol catabolism pathway activator increases expression and/or activity of the iol regulon.

19. The method of claim 1, wherein said myo-inositol catabolism pathway activator is myo-inositol or inositol or a catabolic product thereof.

20. The method of claim 1, wherein said at least 1 agent is at least 2 agents.


SEQUENCE LISTING (14 sequences)

SEQ ID NO: 1 - DNA, 26 bp, Artificial sequence
  Single strand DNA oligonucleotide
tcctgagcctgttgcttaacccggtc

SEQ ID NO: 2 - DNA, 46 bp, Artificial sequence
  Single strand DNA oligonucleotide
caattcgccctatagtgagtcgttccgctcgtccactcccctgctc

SEQ ID NO: 3 - DNA, 56 bp, Artificial sequence
  Single strand DNA oligonucleotide
ccagcttttgttccctttagtgagattcatatgatataatcttaggggtaatagcg

SEQ ID NO: 4 - DNA, 29 bp, Artificial sequence
  Single strand DNA oligonucleotide
cctagtaagaccgtctgtaaacaatacgc

SEQ ID NO: 5 - DNA, 23 bp, Artificial sequence
  Single strand DNA oligonucleotide
atgctagtcggttatacgggatg

SEQ ID NO: 6 - DNA, 51 bp, Artificial sequence
  Single strand DNA oligonucleotide
caattcgccctatagtgagtcgttcggatgtacctcctagaaataaactcg

SEQ ID NO: 7 - DNA, 49 bp, Artificial sequence
  Single strand DNA oligonucleotide
ccagcttttgttccctttagtgagggataccgttcttaaaaaaccaggg

SEQ ID NO: 8 - DNA, 24 bp, Artificial sequence
  Single strand DNA oligonucleotide
gatcggttaggttttcttgttcca

SEQ ID NO: 9 - DNA, 26 bp, Artificial sequence
  Single strand DNA oligonucleotide
cgctttaatttcgggatgctcgagga

SEQ ID NO: 10 - DNA, 51 bp, Artificial sequence
  Single strand DNA oligonucleotide
caattcgccctatagtgagtcgtataaaaaactccttcttgaatctttacg

SEQ ID NO: 11 - DNA, 55 bp, Artificial sequence
  Single strand DNA oligonucleotide
ccagcttttgttccctttagtgagcgtttacaatagtgttgagagtctatcatcc

SEQ ID NO: 12 - DNA, 27 bp, Artificial sequence
  Single strand DNA oligonucleotide
ctgatattttctttgaaacgctcaccc

SEQ ID NO: 13 - protein, 890 aa, Bacillus subtilis
MKFHEMGQTDLLEATNTSMKQGLTEKEVKKRLDKHGPNELQEGKKTSALLLFFAQFKDFMVLVLLAATLISGFLGEYVDAVAIIAIVFVNGILGFFQERRAEQSLQALKELSTPHVMALREGSWTKIPSKELVPGDIVKFTSGDRIGADVRIVEARSLEIEESALTGESIPVVKHADKLKKPDVSLGDITNMAFMGTIVTRGSGVGVVVGTGMNTAMGKIADMLESAGTLSTPLQRRLEQLGKILIVVALLLTVLVVAVGVIQGHDLYSMFLAGVSLAVAAIPEGLPAIVTVALSLGVQRMIKQKSIVRKLPAVETLGCASIICSDKTGTMTQNKMTVTHVWSGGKTWRVAGAGYEPKGSFTLNEKEISVNEHKPLQQMLLFGALCNNSNIEKRDGEYVLDGDPTEGALLTAARKGGFSKEFVESNYRVIEEFPFDSARKMMTVIVENQDRKRYIITKGAPDVLMQRSSRIYYDGSAALFSNERKAETEAVLRHLASQALRTIAVAYRPIKAGETPSMEQAEKDLTMLGLSGIIDPPRPEVRQAIKECREAGIKTVMITGDHVETAKAIAKDLRLLPKSGKIMDGKMLNELSQEELSHVVEDVYVFARVSPEHKLKIVKAYQENGHIVAMTGDGVNDAPAIKQADIGVSMGITGTDVAKEASSLVLVDDNFATIKSAIKEGRNIYENIRKFIRYLLASNVGEILVMLFAMLLALPLPLVPIQILWVNLVTDGLPAMALGMDQPEGDVMKRKPRHPKEGVFARKLGWKVVSRGFLIGVATILAFIIVYHRNPENLAYAQTIAFATLVLAQLIHVFDCRSETSVFSRNPFQNLYLIGAVLSSILLMLVVIYYPPLQPIFHTVAITPGDWMLVIGMSAIPTFLLAGSLLTRKK

SEQ ID NO: 14 - protein, 83 aa, Bacillus subtilis
MTKNQNQYQQPNPDDRSDNVEKLQDMVQNTIENIEEAEASMEFASGEDKQRIKEKNARREQSIEAFRNEIQDESAARQNGYRS